(19) United States
(12) Patent Application Publication
(10) Pub. No.: US 2005/0250837 A1
(43) Pub. Date: Nov. 10, 2005

(54) Use of C-Raf inhibitors for the treatment of neurodegenerative diseases

(76) Inventors: Santosh R. D'Mello, Dallas, TX (US); Paul C. Chin, Plano, TX (US)

Correspondence Address: Scott C. Sample;Locke Liddell & Sapp LLP, 2200 Ross Avenue, Suite 2200, Dallas, TX, 75201-6776, US

(21) Appl. No.: 10/688,759

(22) Filed: Oct. 17, 2003

Related U.S. Application Data
(60) Provisional application No. 60/419,439, filed on Oct. 18, 2002.
(60) Provisional application No. 60/440,177, filed on Jan. 15, 2003.

(51) Int. Cl. A61K 31/404

(52) U.S. Cl. 514/418

(57) ABSTRACT

C-Raf inhibitors, especially oxindole derivatives such as 5-Iodo-3-[(3,5-dibromo-4-hydroxyphenyl) methylene]-2-indolinone, are used for the prevention or inhibition of neuronal cell death in a mammal suffering from or susceptible to neurodegenerative disease, cerebral ischaemia, traumatic neuronal injury, epilepsy-associated neuronal loss, paralysis, or spinal cord injury. C-Raf inhibitors are included in the manufacture of compositions for the treatment of neurodegenerative disease, cerebral ischaemia, traumatic neuronal injury, epilepsy-associated neuronal loss, paralysis, or spinal cord injury.

### CROSS-REFERENCE TO RELATED APPLICATIONS

This application claims the benefit of U.S. Provisional Patent Application No. 60/419,439 filed Oct. 18, 2002 and U.S. Provisional Patent Application No. 60/440,177 filed Jan. 15, 2003.

### GOVERNMENT SUPPORT

This research was supported in part by funds from the Department of Defense (DAMD17-99-1-9566) and the National Institute of Neurological Diseases and Stroke (NS40408).

### TECHNICAL FIELD OF THE INVENTION

This invention is in the field of treating neurodegenerative diseases and conditions. More particularly, this invention is in the field of using C-Raf inhibitors to treat neurodegenerative diseases and conditions.

### BACKGROUND OF THE INVENTION

Neurological diseases disrupt the quality of life for patients, put a tremendous burden on family caregivers, and cost society billions of dollars annually. Increasing numbers of elderly people in the population has resulted in a sharp increase in the prevalence of neurological diseases. Underlying a majority of these diseases is the abnormal degeneration of neurons.

Major aspects of the invention regard mechanisms by which neuronal cells die or survive. Aberrant apoptosis is a common feature in a variety of neurodegenerative diseases, in neuropathological conditions such as stroke and following traumatic brain injury. Much of the knowledge of how neuronal apoptosis is regulated has come from in vitro paradigms using primary cultures of neurons and several molecules involved in promoting neuronal apoptosis have been identified (reviewed in Deshmukh et al., 1997; D'Mello et al., 1998; Mattson, 2000; Chang et al., 2002). Among these is the transcription factor c-jun, which is phosphorylated and activated during the apoptotic process (Estus et al., 1994; Ham et al., 1995; Watson et al. 1998). Phosphorylation of c-jun is mediated by jun N-terminal kinase (JNK; Eilers et al., 1998), which can be encoded by three genes-JNK1, JNK2, and JNK3 (reviewed by Barr and Bogoyevitch, 2001; Weston and Davis, 2002). Although the JNKs can be activated in vitro by MKK4 or MKK7, only MKK7 appears to be involved in stimulating c-jun phosphorylation during neuronal apoptosis (Eilers et al., 1998; Trotter et al., 2002). Members of the mixed lineage kinase (MLK) family lie upstream of MKK4 and MKK7 (Xu et al., 2001; Harris et al, 2002). Besides activating c-jun, JNKs have been shown to activate certain proapoptotic Bcl2 proteins (Harris et al., 2001; Putcha et al., 2003), which contribute to the activation of caspases. Caspases are a family of cysteine proteases known to be critical for cell death in a variety of in vivo and cell culture paradigms of neurodegeneration. Several lines of evidence also implicate an abortive reentry into the cell cycle caused by activation of certain cyclin-dependent kinases (cdks) as a critical feature of neuronal apoptosis (reviewed in Copani et al., 2001; Liu and Greene, 2001; O'Hare et al., 2002).

In the presence of survival-promoting stimuli such as neuronal activity or neurotrophic growth factors, the activation of proapoptotic molecules is blocked. One signaling pathway involved in the promotion of growth factor-mediated neuronal survival is the phosphatidylinositol 3-kinase (PI-3K)-Akt pathway (Datta et al., 1997; Dudek et al., 1997; Crowder et al., 1998). Once activated, Akt phosphorylates a number of proapoptotic molecules including the Bcl-2 protein BAD, the Forkhead transcription factor, glycogen synthase kinase-3 (GSK-3) and caspase-9 (for review, Brunet et al., 2001) leading to their inactivation.

Another signaling pathway that has been implicated in the promotion of neuronal survival is the Raf-MEK-ERK pathway (Villalba et al, 1997; Anderson and Tolkovsky, 1999; Bonni et al., 1999; Mazzoni et al., 1999; Han and Holtzman, 2000). In this pathway, Raf is recruited to the plasma membrane and directly interacts with GTP-Ras. Upon activation, Raf phosphorylates mitogen activated protein kinase (MEK), which in turn phosphorylates and activates extracellular signal-regulated kinases (ERK 1/2). In neuronal populations in which the Raf-MEK-ERK pathway sustains neuronal survival, ERK activation leads to the activation of the CREB transcription factor or the inactivation of BAD (Bonni et al., 1999). Although the ERK pathway is the major effector of Raf, recent evidence suggests that it is not the only one (reviewed in Baccarini, 2002; Hindley and Kolch, 2002). Mammals possess three Raf proteins: C-Raf (also called Raf-1), A-Raf, and B-Raf (reviewed in Baccarini, 2002; Dhillon and Kolch, 2002; Hindley and Kolch, 2002). While C-Raf is expressed ubiquitously, the expression of B-Raf is restricted primarily to the nervous system. Additionally, B-Raf is the most potent activator of MEK and A-Raf is the weakest. Mice deficient in each of the three Raf genes have been generated. While mice deficient in A-Raf are viable albeit with minor gastrointestinal and neurological defects, disruption of either C-Raf or B-Raf results in embryonic lethality. Interestingly, sensory neurons and motoneurons cultured from B-Raf-deficient embryos (but not from C-Raf or A-Raf deficient embryos) fail to survive in response to neurotrophic factors (Weise et al., 2001).

The amount of knowledge that has been recently generated about the molecular regulation of neuronal apoptosis has permitted the rational design of drugs to reduce or prevent neuronal loss in neuropathologies. Much effort towards development of neuroprotective agents has focused on target molecules such as the JNKs, MLKs, cdks, proapoptotic Bcl2 proteins, and the caspases (reviewed in O'Hare et al., 2002; Saporito et al., 2002; Vila and Przedborski, 2003).

Approaches to inhibit neurodegeneration have targeted molecules in the MLK-JNK and cyclin-dependent kinase pathways; however, C-Raf is not directly connected with these two neuronal death-inducing signaling pathways. C-Raf (and A-Raf and B-Raf) has not been implicated in the promotion of cell death in neurons or any other cell type and is not a component of any cell-death inducing signal transduction pathway. On the contrary, elevated activity of C-Raf (and other Rafs) have is known to enhance cell survival and an over activation of C-Raf contributes to cell transformation and cancer. Thus, the present discovery that inhibition of C-Raf has neuroprotective effects is counterintuitive and is novel.

### SUMMARY OF THE INVENTION

In accordance with the present invention, the use of compounds as neuroprotectors and a method for the treatment of neurodegenerative diseases is provided. The present invention provides for neuroprotection effected by C-Raf inhibition. C-Raf inhibitors are used in the treatment of and the manufacture of compositions for treatment of neurodegenerative disease, traumatic neuronal injury, epilepsy-associated neuronal loss, paralysis, or spinal cord injury. The present invention provides C-Raf inhibitors used to prevent neuronal death. The present invention also provides for the use of C-Raf inhibitors to prevent neuronal cell death potentiated by inhibition or suppression of B-Raf. The present invention further provides for the use of C-Raf inhibitors derived from oxindoles to prevent neuronal cell death and in the treatment of neural diseases, injuries, paralyses and other aspects of abnormal neural function. In addition to the use of C-Raf inhibitors in the manufacture of compositions for the treatment of various aspects of abnormal neural function, methods of using C-Raf inhibitors to treat neurodegenerative diseases and conditions are also provided.

### BRIEF DESCRIPTION OF THE DRAWINGS

A more particular description of the invention, briefly summarized above, may be had by reference to the embodiments thereof which are illustrated in the appended drawings and described herein. It is to be noted, however, that the appended drawings and tables illustrate only some embodiments of the invention and are therefore not to be considered limiting of its scope, because the invention may admit to other equally effective embodiments.

FIGS. 1A-1C are phase contrast micrographs and FIG. 1D is a graph showing the neuroprotective effect of the C-Raf inhibitor, GW5074, according to one embodiment of the invention.

FIGS. 2A-2C are Western blots showing that a highly neuroprotective C-Raf inhibitor nonetheless permits activating modifications to accumulate in intact neurons.

FIG. 3A is a Western blot and FIG. 3B is a graph showing the neuroprotective effect of a C-Raf inhibitor according to another embodiment of the invention.

FIG. 4 is a Western blot showing that GW5074 activates B-Raf in neurons and leads to ERK phosphorylation.

FIG. 5A is a Western blot and FIG. 5B is a graph showing that GW5074-mediated neuroprotection is MEK-ERK independent.

FIGS. 6A-B is a Western blot and FIG. 6C is a graph showing that GW5074 maintains Akt activity but acts through an Akt-independent mechanism FIGS. 7A and 7C are graphs and FIG. 7B is a gel mobility shift assay blot showing that neuroprotection by GW5074 requires Ras and NF-κB.

FIG. 8 is a Western blot showing that GW5074 inhibits apoptosis-associated induction of c-jun.

FIGS. 9A-9B are graphs showing that GW5074 inhibits cell death caused by neurotoxins in granule cells and other neuronal types.

FIGS. 10A-10C are light and phase contrast micrographs showing that GW5074 is protective in an in vivo experimental model of Huntington's disease.

### DETAILED DESCRIPTION OF A PREFERRED EMBODIMENT

The discussion and examples which follow detail the best known method for performing the invention. It will be recognized that variations of this method may include different C-Raf inhibitors, different neuronal populations, and different neurodegenerative diseases; however, other C-Raf inhibitors may be identified to treat neurodegenerative diseases without significant experimentation or deviation from the spirit and scope of this invention. In view of the involvement of B-Raf in the neuroprotective action of C-Raf inhibitors, activators of B-Raf could be used to treat neurodegenerative conditions.

Definitions

“Neurodegeneration” refers to the compromised function or death of cells within the peripheral nervous system or the central nervous system.

“Neurodegenerative diseases or conditions” refers to pathological conditions affecting the peripheral nervous system or the central nervous system and characterized by an abnormal loss of neural cells. Such conditions include neurodegenerative diseases including Alzheimer's disease, Parkinson's disease, Huntington's disease, Amyotrophic Lateral Sclerosis, cerebral ischaemia, ataxias, epilepsy-associated neuronal loss, traumatic neuronal or spinal cord injury or neurotoxicity, which may be caused by genetic factors or environmental stimuli, or both.

Apoptosis or apoptotic, as defined herein, refers to a programmed cell death with characteristic morphological and biochemical features known to those skilled in the art (see for example Oppenheim 1991 and Johnson & Deckworth 1993; and references cited therein).

“C-Raf inhibitors” refers to chemical or biological agents that reduce or inhibit the activity of the C-Raf kinase. As illustrative only, and not to exclude the use of other C-Raf inhibitors, some preferred C-Raf inhibitors include the family of compounds having the general structural formula (I): 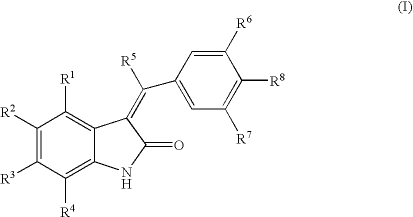
wherein:
R¹ is H or optionally joined with R² to form a fused ring selected from the group consisting of five to ten membered aryl, heteroaryl or heterocyclyl rings, said heteroaryl or said heterocyclyl rings having one to three heteroatoms where zero to three of said heteroatoms are N and zero to 1 of said heteroatoms are O or S and where said fused ring is optionally substituted by one to three of R⁹, where R² and R⁹ are as defined below;
R² and R³ are independently H, HET, aryl, C₁₋₁₂ aliphatic, CN, NO₂, halogen, R¹⁰, —OR¹⁰, —SR¹⁰, —S(O)R¹⁰, —SO₂R¹⁰, —NR¹⁰R¹¹, —NR¹¹R¹², —NR¹²COR^{11,} —NR¹²CO₂R¹¹, —NR¹²CONR¹¹R¹², —NR¹²SO₂R¹¹, —NR¹²C(NR¹²)NHR¹¹, —COR¹¹, —CO₂R¹¹, —CONR¹²R¹¹, —SO₂NR¹²R¹¹, —OCONR¹²R¹¹, C(NR¹²)NR¹²R¹¹
where said C₁₋₁₂ aliphatic optionally bears one or two insertions of one to two groups selected from C(O), O, S, S(O), SO₂ or NR¹²; with said HET, aryl or C₁₋₁₂ aliphatic being optionally substituted by one to three of R¹⁰; and where R² is optionally joined with R³ to form a fused ring selected from the group consisting of five to ten membered aryl, heteroaryl or heterocyclyl rings, said heteroaryl or said heterocyclyl rings having zero to three heteroatoms where zero to three of said heteroatoms are N and zero to one of said heteroatoms are O or S and where said fused ring is optionally substituted by one to three of R⁹, where HET, R⁹, R¹⁰, R¹¹ and R¹² are as defined below;
R⁴ is H, halogen, NO₂ or CN;
R⁵ is H or C₁₋₁₂ aliphatic optionally substituted by one to three of halo, hydroxyl, heteroaryl, or aryl;
R⁶ and R⁷ are independently halogen, CN, NO₂, —CONR¹⁰R¹¹, —SO₂NR¹⁰R¹¹, —NR¹⁰R¹¹, or —OR¹¹, where R¹⁰ and R¹¹ are as defined below;
R⁸ is OH, NHSO₂R¹² or NHCOCF₃;
R⁹ is each independently halogen, C₁₋₁₂ aliphatic, CN, —NO₂, R¹⁰, —OR¹¹, —SR¹¹, —S(O)R¹⁰, —SO₂R¹⁰, —NR¹⁰R¹¹, —N¹¹R¹², —NR¹²CO₂R¹¹, —NR¹²CO₂R¹¹, —NR¹²CONR¹¹R¹², —NR¹²SO₂R¹¹, —NR¹²C(NR¹²C)NHR¹¹, —CO₂R¹¹, CONR¹²R¹¹, —SO₂NR¹²R¹¹, —OCONR¹²R¹¹ or C(NR¹²)NR¹²R¹¹, where R¹⁰, R¹¹ and R¹² are as defined below;
R¹⁰ is each independently H, halogen, C₁₋₁₂ aliphatic, aryl or HET, where said C₁₋₁₂ aliphatic optionally bears an inserted one to two groups selected from O, S, S(O), SO₂ or NR¹², where said C₁₋₁₂ aliphatic, aryl or HET is optionally substituted by one to three of halo, another HET, aryl, CN, —SR¹², —OR¹², —N(R¹²)₂, —S(O)R¹², —SO₂R¹², —SO₂N(R¹²)₂, —NR¹²COR¹², —NR¹²CO₂R¹², —NR¹²CON(R¹²)₂, —NR¹²(NR¹²)NHR¹², —CO₂R¹², —CON(R¹²)₂, —NR¹²SO₂R¹², —OCON(R¹²)₂, where HET and R¹² are as defined below;
R¹¹ is H or R¹⁰;
R¹² is H, C₁₋₁₂ aliphatic or HET, said C₁₋₁₂ aliphatic optionally substituted by one to three of halogen or OH where HET is as defined below; and
HET is a five-to ten-membered saturated or unsaturated heterocyclic ring selected from the group consisting of benzofuran, benzoxazole, dioxin, dioxane, dioxolane, dithiane, dithiazine, dithiazoie, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thidiazole, thiatriazole, thiazine, thiazole, thiomorpholine, thianaphthalene, thiopyran, triazine, and triazole;
and the pharmaceutically acceptable salts, biohydrolyzable esters, biohydolyzable amides, biohydrolyzable carbamates, biohydrolyzable carbonates, biohydrolyzable ureides, solvates, hydrates, affinity reagents or prodrugs of (I) as defined above.

A preferred group of compounds of the present invention are those of the general formula (I): 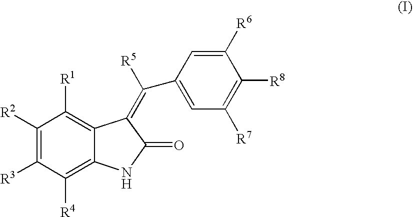
wherein R¹ is H or optionally joined with R² to form a fused ring selected from the group as defined for HET below, and where said fused ring is optionally substituted by one to three of R⁹, where R² and R⁹ are as defined below;
R² and R³ are independently H, HET, aryl, C₁₋₆ aliphatic, CN, NO₂, halogen, R¹⁰, —OR¹⁰, —SR¹⁰, —S(O)R¹⁰, —SO₂R¹⁰, —NR¹⁰R¹¹, —NR¹¹R¹², —NR¹²COR¹¹, —NR¹²CO₂R¹¹, —NR¹²CONR¹¹R¹², —NR¹²SO₂R¹¹, —NR¹²C(NR¹²)NHR¹¹, —COR¹¹, —CO₂R¹¹, —CONR¹²R¹¹, —SO₂NR¹²R¹¹, —OCONR¹²R¹¹, C(NR¹²)NR¹²R¹¹ where said C₁₋₆ aliphatic optionally bears one or two insertions of one to two groups selected from C(O), O, S, S(O), SO₂, or NR¹², with said HET, aryl or C₁₋₆ aliphatic being optionally substituted by one to three of R¹⁰; and where R² is optionally joined with R³ to form a fused ring selected from the group as defined below and where said fused ring is optionally substituted by one to three of R⁹, where HET, R⁹, R¹⁰, R¹¹ and R¹² are as defined below;
R⁴ is H, halogen, NO₂ or CN;
R⁵ is H or C₁₋₆ aliphatic optionally substituted by one to three of halo, OH, or aryl;
R⁶ and R⁷ are independently halogen, CN, NO₂, —CONR¹⁰R¹¹, —SO₂NR¹⁰R¹¹, —NR¹¹OR¹¹, or —OR¹¹, where R¹⁰ and R¹¹ are as defined below;
R⁸ is OH, NHSO₂R¹² or NHCOCF₃;
R⁹ is each independently halo, C₁₋₆ aliphatic, CN, —NO₂, R¹⁰, —OR¹¹, —SR¹¹, —S(O)R¹⁰, —SO₂R¹⁰, —NR¹⁰R¹¹, —N¹¹R¹², —NR¹²COR¹¹, —NR¹²CO₂R¹¹, —NR¹²CONR¹¹R¹², —NR¹²SO₂R¹¹, —NR¹²C(NR¹²)NHR¹¹, —CO₂R¹¹, —CONR¹²R¹¹, —SO₂NR¹²R¹¹, —OCONR¹²R¹¹ or C(NR¹²)NR¹²R¹¹, where R¹⁰, R¹¹ and R¹² are as defined below;
R¹⁰ is each independently H, halogen, C₁₋₆ aliphatic, aryl or HET, where said C₁₋₆ aliphatic optionally bears an inserted one to two groups selected from O, S, S(O), SO₂ or NR², where said C₁₋₆ aliphatic, aryl or HET is optionally substituted by one to three of halo, another HET, aryl, CN, —SR¹², —R¹², —N(R¹²)₂, —S(O)R², —SO₂R¹², —SO₂N(R²)₂, —NR²COR², —NR²CO₂R², —NR²CON(R²)₂, —NR(NR¹²)NHR¹², —CO₂R¹², —CON(R¹²)₂, —NR¹²SO₂R¹², —OCON(R¹²)₂, where HET and R¹² are as defined below;
R¹¹ is H or R¹⁰;
R¹² is H, C₁₋₆ aliphatic or HET, said C₁₋₆ aliphatic optionally substituted by one to three of halogen or OH where HET is as defined below; and
HET is a five to ten-membered saturated or unsaturated heterocyclic ring selected from the group consisting of benzofuran, benzoxazole, dioxin, dioxane, dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole;
and the pharmaceutically acceptable salts, biohydrolyzable esters, biohydolyzable amides, biohydrolyzable carbamates, biohydrolyzable carbonates, biohydrolyzable ureides, solvates, hydrates, affinity reagents or prodrugs of (I) as defined above.

A highly preferred group of compounds of the present invention are those of the general formula (I): 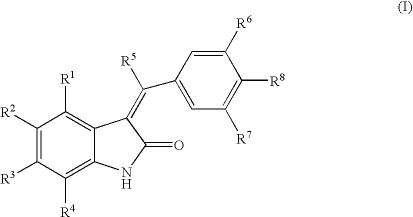
wherein R¹ is H or optionally joined with R² to form a fused ring selected from the group consisting of fused pyridine, fused triazole, fused thiazole or fused amino-substituted thiazole;
R² and R³ are independently H, HET, aryl, C₁₋₆ aliphatic, —R¹²NH₂, —R¹² halogen, CN, NO₂, halogen, R¹⁰, —OR¹⁰, —SR¹⁰, —S(O)R¹⁰, —SO₂R¹⁰, —NR¹⁰R¹¹, —NR¹¹R¹², —NR¹²COR¹¹, —NR¹²CO₂R¹¹, —NR¹²CONR¹¹R¹², —NR¹²SO₂R¹¹, —NR¹²C(NR¹²)NHR¹¹, —COR¹¹, —COR¹¹ NR¹²R¹¹—CO₂R¹¹, —CONR¹²R¹¹, —SO₂NR¹²R¹¹, —OCONR¹²R¹¹, —C(NH)R¹¹, —C(NR¹²)NR¹²R¹¹ where said C₁₋₆ aliphatic optionally bears an insertion of a C(O) group; with said HET, aryl or C₁₋₆ aliphatic being optionally substituted by one to three of R¹⁰; and where R² is optionally joined with R³ to form a fused ring selected from the group as defined for HET below and where said fused ring is optionally substituted by one to three of R⁹, where HET, R⁹, R¹⁰, R¹¹ and R¹² are as defined below;
R⁴ is H, halogen, NO₂ or CN;

R⁵ is H or C₁₆ aliphatic optionally substituted by one to three of halogen, OH, or aryl;
R⁶ and R⁷ are independently halogen, CN, NO₂, —CONR¹⁰R¹¹, —SO₂NR¹⁰R¹¹, —NR¹⁰R¹¹, or —OR¹¹, where R¹⁰ and R¹¹ are as defined below;
R⁸ is OH, NHSO₂R¹² or NHCOCF₃;
R⁹ is each independently halo, C₁₋₆ aliphatic, CN, —NO₂, R¹⁰, —OR¹¹, —SR¹¹, —S(O)R¹⁰, —SO₂R¹⁰, —NR¹⁰R¹¹, —N¹¹R¹², —N¹¹R²COR¹¹, —NR¹²CO₂R¹¹, —NR¹²CONR¹¹R¹², —NR¹²SO₂R¹¹, —NR¹²C(NR¹²), NHR¹¹, —CO₂R¹¹, —CONR¹²R¹¹, —SO₂NR¹²R¹¹, —OCONR¹²R¹¹ or C(NR¹²)NR¹²R¹¹, where R¹⁰, R¹¹ and R¹² are as defined below;
R¹⁰ is each independently H, halogen, C₁₋₆ aliphatic, aryl or HET, where said C₁₋₆ aliphatic optionally bears an inserted one to two groups selected from O, S, S(O), SO₂ or NR¹², where said C₁₋₆ aliphatic, aryl or HET is optionally substituted by one to three of halo, another HET, aryl, CN, NO₂—R¹², —SR¹², —OR², —N(R¹²)₂, —R¹²N(R²)₂—S(O)R¹², —SO₂R², —SO₂N(R¹²)₂, —NR¹²COR¹², —NR¹²CO₂R¹², —NR¹²CON(R¹²)₂, —NR¹²(NR¹²)NHR¹², —CO₂R¹², —CON(R¹²)₂, —NR¹²SO₂R¹², —OCON(R¹²)₂, or trifluoro, where HET and R¹² are as defined below;
R¹¹ is H or R¹⁰;
R¹² is H, C₁₋₆ aliphatic, NO₂, C₁₋₆ alkoxy, halogen, aryl or HET, said C₁₋₆ aliphatic optionally substituted by one to three of halogen or OH where HET is as defined below;

HET is a five or six-membered saturated or unsaturated heteroaryl ring selected from the group consisting of dioxin, dioxane, dioxolane, dithiane, dithiazine, dithiazole, dithiolane, furan, imidazole, imidazopyridinyl, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, tetrahydrofuran, tetrazine, thiophene, thiadiazine, thiadiazole, thiatriazole, thiazine, thiazole, thiomorpholine, thiopyran, thioxotriazine, triazine, and triazole;
and the pharmaceutically acceptable salts, biohydrolyzable esters, biohydolyzable amides, biohydrolyzable carbamates, biohydrolyzable carbonates, biohydrolyzable ureides, solvates, hydrates, affinity reagents or prodrugs of (I) as defined above.

Also highly preferred is a compound of formula (I) in which R¹ and R² additionally comprise a fused ring which is methyl substituted fused pyridine.

A group of compounds that are preferred with respect to their substitutes at positions R⁶, R⁷ and R⁸ are compounds of the formula: 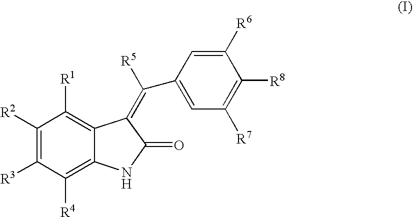
wherein:
R¹ is H or optionally joined with R² to form a fused ring selected from the group consisting of five to ten membered aryl, heteroaryl or heterocyclyl rings, said heteroaryl or said heterocyclyl rings having one to three heteroatoms where zero to three of said heteroatoms are N and zero to 1 of said heteroatoms are O or S and where said fused ring is optionally substituted by one to three of R⁹, where R² and R⁹ are as defined below;
R² and R³ are independently H, HET, aryl, C₁₋₁₂ aliphatic, CN, NO₂, halogen, R¹⁰, —OR¹⁰, —SR¹⁰, —S(O)R¹⁰, —SO₂R¹⁰, —NR¹⁰R¹¹, —NR¹¹R¹², NR¹²COR¹¹, —NR¹²CO₂R¹¹, —NR¹²CONR¹¹R¹², —NR¹²SO₂R¹¹, —NR¹²C(NR¹²)NHR¹¹, —COR¹¹, —CO₂R¹¹, —CONR¹²R¹¹, —SO₂NR¹²R¹¹, —OCONR¹²R¹¹, C(NR¹²)NR¹²R¹ where said C₁₋₁₂ aliphatic optionally bears one or two insertions of one to two groups selected from C(O), O, S, S(O), SO₂ or NR¹²; with said HET, aryl or C₁₋₁₂ aliphatic being optionally substituted by one to three of R¹⁰; and where R² is optionally joined with R³ to form a fused ring selected from the group consisting of five to ten membered aryl, heteroaryl or heterocyclyl rings, said heteroaryl or said heterocyclyl rings having zero to three heteroatoms where zero to three of said heteroatoms are N and zero to one of said heteroatoms are O or S and where said fused rings is optionally substituted by one to three of R⁹, where HET, R⁹, R¹⁰, R¹¹ and R¹² are defined below;
R4 is H, halogen, NO₂ or CN;
R⁵ is H or C₁₋₁₂ aliphatic optionally substituted by one to three of halo, hydroxyl, or aryl;
R⁶ and R⁷ are halogen;
R⁸ is OH;
R⁹ is each independently halogen, C₁₋₁₂ aliphatic, CN, —NO₂, R¹⁰, —OR¹¹—SR¹¹, —S(O)R¹⁰, —SO₂R¹⁰, —NR¹⁰R¹¹, —N¹¹R¹², —NR¹²COR¹¹, —NR¹²CO₂R¹¹, —NR¹²CONR¹¹R¹², —NR¹²SO₂R¹¹, —NR¹²C(NR¹²)NHR¹¹, —CO₂R¹¹, —CONR¹²R¹, —SO₂NR¹²R¹¹, —OCONR²R¹¹ or C(NR¹²)NR¹²R¹¹, where R¹⁰, R¹¹ and R¹² are as defined below;
R¹⁰ is each independently H, halogen, C₁₋₁₂ aliphatic, aryl or HET, where said C₁₋₁₂ aliphatic optionally bears an inserted one to two groups selected from O, S, S(O), SO₂ or NR², where said C₁₋₁₂ aliphatic, aryl or HET is optionally substituted by one to three of halo, another HET, aryl, CN, —SR¹², —OR¹², —N(R¹²)₂, —S(O)R¹², —SO₂R¹², —SO₂N(R¹²)₂, —NR¹²COR¹², —NR¹²CO₂R¹², —NR¹²CON(R¹²)₂, —NR¹²(NR¹²)NHR¹², —CO₂R¹², —CON(R¹²)₂, —NR²S₂R², —OCON(R¹²)₂, where HET and R¹² are as defined below;
R¹¹ is H R¹⁰;
R¹² is H, C₁₋₁₂ aliphatic or HET, said C₁₋₁₂ aliphatic optionaly substituted by one to three of halogen or OH where HET is as defined below; and
HET is a five to ten-membered saturated or unsaturated heterocyclic ring selected from the group consisting of benzofuran, benzoxazole, dioxin, dioxane dioxalane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazxine, pyrazole, pyridine, pyrimidine, pyrrole, pyrrolidine, quinoline, quinazolline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole;
and the pharmaceutically acceptable salts, biohydrolyzable esters, biohydolyzable amides, biohydolyzable, carbamates, biohydrolyzable carbonates, biohydrolyzable ureides, solvates, hydrates, affinity reagents or prodrugs of (I) as defined above.

Another group of compounds that are preferred with respect to their substituents at positions R⁶, R⁷ and R⁸ are compounds of the formula: 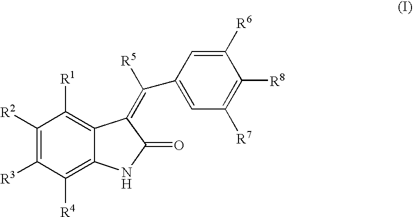
wherein:

R¹ is H or optionally joined with R² to form a fused ring selected from the group consisting of five to ten membered aryl, heteroaryl or heterocyclyl rings, said heteroaryl or said heterocyclyl rings having one to three heteroatoms where zero to three of said heteroatoms are N and zero to 1 of said heteroatoms are O or S and where said fused ring is optionally substituted by one to three of R⁹, where R² and R⁹ are as defined below;
R² and R³ are independently H, HET, aryl, C₁₋₁₂ aliphatic, CN, NO₂, halogen R¹⁰, —OR¹⁰, —SR¹⁰, —S(O)R¹⁰, —SO₂R¹⁰, —NR¹⁰R¹¹, —NR¹¹R¹², NR¹²COR¹¹, —NR¹²CO₂R¹¹, —NR¹²CONR¹¹R¹², —NR¹²SO₂R¹—NR¹²C(NR¹²)NHR¹¹, —COR¹¹, —CO₂R¹¹, —CONR¹²R¹¹, —SO₂NR¹²R¹¹, —OCONR¹²R¹¹, C(NR¹²)NR¹²R¹¹ where said C₁₋₁₂ aliphatic optionally bears one or two insertions of one to two groups selected from C(O), O, S, S(O), SO₂ or NR¹²; with said HET, aryl or C₁₋₁₂ aliphatic being optionally substituted by one to three of R¹⁰; and where R² is optionally joined with R³ to form a fused ring selected from the group consisting of five to ten membered aryl, heteroaryl or heterocyclyl rings, said heteroaryl or said heterocyclyl rings having zero to three heteroatoms where zero to three of said heteroatoms are N and zero to one of said heteroatoms are O or S and where said fused ring is optionally substituted by one to three of R⁹, where HET, R⁹, R¹⁰, R¹¹ and R¹² are as defined below;
R4 is H, halogen, NO₂ or CN;
R5 is H or C¹⁻¹² aliphatic optionally substituted by one to three of halo, hydroxyl, or aryl;
R⁶ and R⁷ are independently bromo or chloro;
R⁸ is OH;
R⁹ is each independently halogen, C₁₋₁₂ aliphatic, CN, —NO₂, R¹⁰, —OR¹¹, —SR¹¹, —S(O)R¹⁰, —SO₂, R¹⁰, —NR¹⁰R¹¹, —N¹¹R¹², —NR¹²COR¹¹, —NR¹²CO₂R¹¹, —NR¹²CONR¹¹R¹², —NR¹²SO₂R¹, —NR¹²C(NR¹²)NHR¹¹, —CO₂R¹¹, —CONR¹²R¹¹, —SO₂NR¹²R¹¹, —OCONR¹²R¹¹ or C(NR¹²)NR¹²R¹¹, where R¹⁰, R¹¹ and R¹² are as defined below;
R¹⁰ is each independently H, halogen, C₁₋₁₂ aliphatic, aryl or HET, where said C₁₋₁₂ aliphatic optionally bears an inserted one to two groups selected from O, S, S(O), SO₂ or NR¹², where said C₁₋₁₂ aliphatic, aryl or HET is optionally substituted by one to three of halo, another HET, aryl, CN, —SR¹², —OR², —N(R¹²)₂, —S(O)R¹², SO₂R¹², —SO₂N(R¹²)₂, —NR¹²COR¹², —NR¹²C₂R¹², —NR¹²CON(R¹²)₂, NR¹² (NR¹²NHR¹², —CO₂R¹², —CON(R¹²)₂, —NR¹²SO₂R¹², —OCON(R¹²)₂, where HET and R¹² are as defined below;
R¹¹ is H or R¹⁰;

R¹² is H, C₁₋₁₂ aliphatic or HET, said C₁₋₁₂ aliphatic optionally substituted aby one to three of halogen or OH where HET is as defined below; and
HET is a five to ten-membered saturated or unsaturated heterocyclic ring selected from the group consisting of benzofuran, benzoxazole, dioxin, dioxane dioxolane, dithiane, dithiazine, dithiazole, dithiolane, furan, imidazole, indole, indazole, morpholine, oxazole, oxadiazole, oxathiazole, oxathiazolidine, oxazine, oxiadiazine, piperazine, piperidine, pyran, pyrazine, pyrazole, pyridine, pyrimidine, pyrrole, pyrrolidine, quinoline, quinazoline, tetrahydrofuran, tetrazine, tetrazole, thiophene, thiadiazine, thiadiazole, thiatriazole, thiazine, thiazole, thiomorpholine, thianaphthalene, thiopyran, triazine, and triazole;
and the pharmaceutically acceptable salts, biohydrolyzable esters, biohydolyzable amides, biohydrolyzable carbamates, biohydrolyzable carbonates, biohydrolyzable ureides, solvates, hydrates, affinity reagents or prodrugs of (I) as defined above.

Yet another group of compounds that are preferred with respect to their substituents at positions R⁶, R⁷ and R⁸ are compounds of the formula: 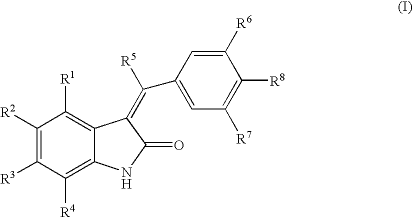
wherein

R¹ is H or optionally joined with R² to form a fused ring selected from the group consisting of five to six membered heteroaryl rings, said heteroaryl ring having one to two heteroatoms where zero to two of said heteroatoms are N and zero to two of said heteroatoms are O or S and where said fused ring is optionally substituted by one to three of R⁹, where R² and R⁹ are as defined below;
R² and R³ are independently H, HET, phenyl, C₁₋₆ aliphatic, —NR¹⁰R¹¹, —COR¹¹, —CO₂R¹¹, —CONR¹²R¹¹, —SO₂NR¹²R¹¹, with said HET, phenyl or C₁₋₆ aliphatic being optionally substituted by R¹⁰; and where R² is optionally joined with R³ to form a fused five membered heterocyclyl ring, said heterocyclyl ring having zero to 1 heteroatoms where said heteroatom is N and zero to 1 heteroatoms where said heteroatoms are O or S and where said fused ring is optionally substituted by R⁹, where HET, R⁹, R¹⁰, R¹¹ and R¹² are as defined below;
R⁴ is H;
R⁵ is H;
R⁶ and R⁷ are independently bromo or chloro;

R⁸ is OH;
R⁹ is H, C₁₋₆ aliphatic, or —COR¹⁰, where R¹⁰ is as defined below;
R¹⁰ is H, C₁₋₆ aliphatic or amino;
R¹¹ is H, C₁₋₆ aliphatic, hydroxy-C₁₋₆ aliphatic, phenyl, phenyl-C₁₋₆ aliphatic or HET;
R¹² is H, C₁₆ aliphatic, hydroxy-C₁₋₆ aliphatic or (R¹¹)₂ N—C₁₋₆ aliphatic; and
HET is a heterocyclic ring selected from the group consisting of oxazole, pyridine, tetrazole and thiazole;
and the pharmaceutically acceptable salts, biohydrolyzable esters, biohydolyzable amides, biohydrolyzable carbamates, biohydrolyzable carbonates, biohydrolyzable ureides, solvates, hydrates, affinity reagents or prodrugs of (I) as defined above.

Still another group of compounds that are preferred with respect to their substituents at positions R⁶, R⁷ and R⁸ are compounds of the formula: 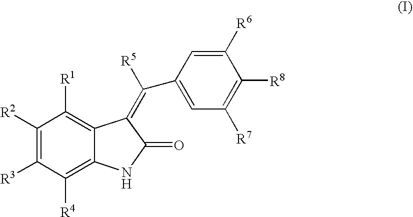
wherein
R¹ is H;
R² and R³ are independently H, HET, phenyl, C₁₋₆, aliphatic, cyano, halogen, —COR¹¹, or —CONR¹²R¹¹, with said HET, phenyl or C₁₋₆, aliphatic being optionally substituted by R¹⁰, where HET, R¹⁰, R¹¹ and R¹² are as defined below;
R⁴ is H;
R⁵ is H;
R⁶ and R⁷ are independently bromo or chloro;
R⁸ is OH;
R¹⁰ is H, C₁₋₆ aliphatic, oxo or cyano;
R¹¹ is H, C₁₆ aliphatic, trihalo-C₁₋₆ aliphatic, phenyl or nitro-substituted phenyl;

R¹² is H, C₁₋₆ aliphatic, hydroxy-C₁₋₆ aliphatic; and
HET is thiophene or pyridine;
and the pharmaceutically acceptable salts, biohydrolyzable esters, biohydolyzable amides, biohydrolyzable carbamates, biohydrolyzable carbonates, biohydrolyzable ureides, solvates, hydrates, affinity reagents or prodrugs of formula (I) as defined above.

Certain compounds of formula (I) above may exist in stereoisomeric forms (e.g. they may contain one, or more asymmetric carbon atoms or may exhibit cis-trans isomerism). The individual stereoisomers (enantiomers and diastereoisomers) and mixtures of these are included within the scope of the present invention. Likewise, it is understood that compounds of formula (I) may exist in tautomeric forms other than that shown in the formula and these are also included within the scope of the present invention.

Due to the presence of a double bond, also included in the compounds of the invention are their respective pure E and Z geometric isomers as well as mixtures of E and Z isomers.

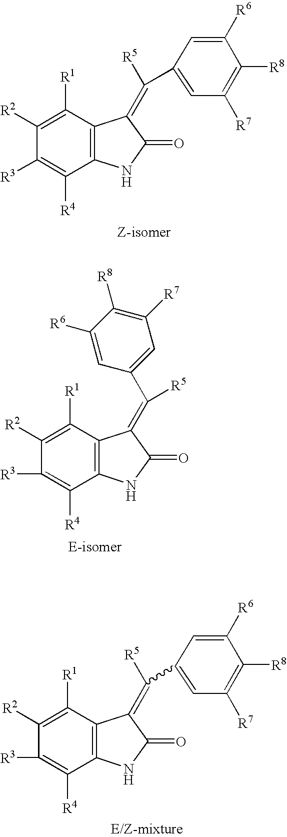

E/Z Mixture

The invention as described and claimed does not set any limiting ratios on prevalence of Z to E isomers.

Certain of the compounds as described will contain one or more chiral carbons and will therefore be either dextrorotatory or levorotatory. Also included in the compounds of the invention are the respective dextrorotatory or levorotatory pure preparations, and racemic mixtures thereof.

Salts of the compounds of the present invention may comprise acid addition salts derived from a nitrogen on a substituent in the compound of formula (I). The therapeutic activity resides in the moiety derived from the compound of the invention as defined herein and the identity of another component is of less importance although for therapeutic and prophylactic purposes it is, preferably, pharmaceutically acceptable to the patient.

Highly preferred biohydrolyzable carbamates comprise compounds of formula (i), wherein R⁸ is OH and said OH is conjugated with a carbamoyl conjugate to yield a biohydrolyzable carbamate wherein said carbamoyl conjugate is selected from the group consisting of diethylaminocarbonyl, N-(2-hydroxyethyl)aminocarbonyl, N,N,-bis(2-hydroxyethyl)aminocarbonyl, hydroxyethyloxyethylaminocarbonyl, 4-morpholinocarbonyl and 4-methyl-1-piperazinylcarbonyl.

Highly preferred biohydrolyzable carbonates comprise compounds of formula (I), where R⁸ is OH and said OH is conjugated with a carbonate conjugate to yield a biohydrolyzable carbonate wherein said carbonyl conjugate is selected from the group consisting of phenylmethyloyxcarbonyl, ethyloxycarbonyl, isobutyloxycarbonyl, and pyridinemethyloxycarbonyl.

A discussion of suitable C-Raf inhibitors is found in U.S. Pat. No. 6,268,391, the disclosure of which is hereby incorporated by reference.

Formulation and Dosing

In order to use C-Raf inhibitors and B-Raf activators in therapy, they will normally be formulated into a pharmaceutical composition in accordance with standard pharmaceutical practice.

C-Raf inhibitors and B-Raf activators may conveniently be administered by any of the routes conventionally used for drug administration, for instance, parenterally, orally, topically or by inhalation. C-Raf inhibitors and B-Raf activators may be administered in conventional dosage forms prepared by combining then with standard pharmaceutical carriers according to conventional procedures. C-Raf inhibitors and B-Raf activators may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable carrier is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquid such as an ampoule or nonaqueous liquid suspension.

C-Raf inhibitors and B-Raf activators are preferably administered parenterally, that is by intravenous, intramuscular, subcutaneous intranasal, intrarectal, intravaginal or intraperitoneal administration. The intravenous form of parenteral administration is generally preferred. Appropriate dosage forms for such administration may be prepared by conventional techniques.

C-Raf inhibitors and B-Raf activators may also be administered orally. Appropriate dosage forms for such administration may be prepared by conventional techniques.

C-Raf inhibitors and B-Raf activators may also be administered by inhalation, that is by intranasal and oral inhalation administration. Appropriate dosage forms for such administration, such as aerosol formulations, may be prepared by conventional techniques.

C-Raf inhibitors and B-Raf activators may also be administered topically, that is by non-systemic administration. This includes the application of the C-Raf inhibitors and B-Raf activators externally to the epidermis or the buccal cavity and the instillation of such a compound into the ear, eye and nose, such that the compound does not significantly enter the blood stream.

For all methods of use disclosed herein for C-Raf inhibitors and B-Raf activators, especially oxindole derivatives, the daily oral dosage regimen can optionally be from about 0.1 to about 80 mg/kg of total body weight, preferably from about 0.2 to 30 mg/kg, more preferably from about 0.5 mg to 15 mg/kg. The daily parenteral dosage regimen can optionally be about 0.1 to about 80 mg/kg of total body weight, preferably from about 0.2 to about 30 mg/kg, and more preferably from about 0.5 mg to 15 mg/kg. The daily topical dosage regimen can optionally be from 0.1 mg to 150 mg/kg, administered one to four, preferably two or three times daily. The daily inhalation dosage regimen can optionally be from about 0.01 mg/kg to about 1 mg/kg per day.

It will also be recognized by one of skill in the art that the optimal quantity and spacing of individual dosages of the C-Raf inhibitors and B-Raf activators will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of the C-Raf inhibitors and B-Raf activators given per day for a defined number of days, can be ascertained by those skilled in the art using conventional course of treatment determination tests.

In all aspects of the invention, the use/method/agonist/medicament can involve delayed administration to a mammal of an effective amount of a C-Raf inhibitor or B-Raf activator, or a pharmaceutically acceptable salt, complex or prodrug thereof, after an acute neurodegenerative or potentially neurodegenerative occurrence, for example traumatic or mechanical neuronal injury or cerebral ischaemia. The time of administration can be 30 or 60 minutes or more after the said occurrence, and/or can be up to 8 or 6 or 4 or 2 or 1 hour(s) after the said occurrence, e.g. 30 mins to 8 hours, 30 mins to 6 hours, or 30 mins to 4 hours after said occurrence. C-Raf inhibitor or B-Raf activator might be neuroprotective when administered within these time frames after such occurrences, which would allow administration in hospital after the occurrence.

All publications, including but not limited to patents and patent applications, cited in this specification are herein incorporated by reference as if each individual publication were specifically and individually indicated to be incorporated by reference herein as though fully set forth.

Embodiments of the present invention are set forth in the Examples below.

### EXAMPLES

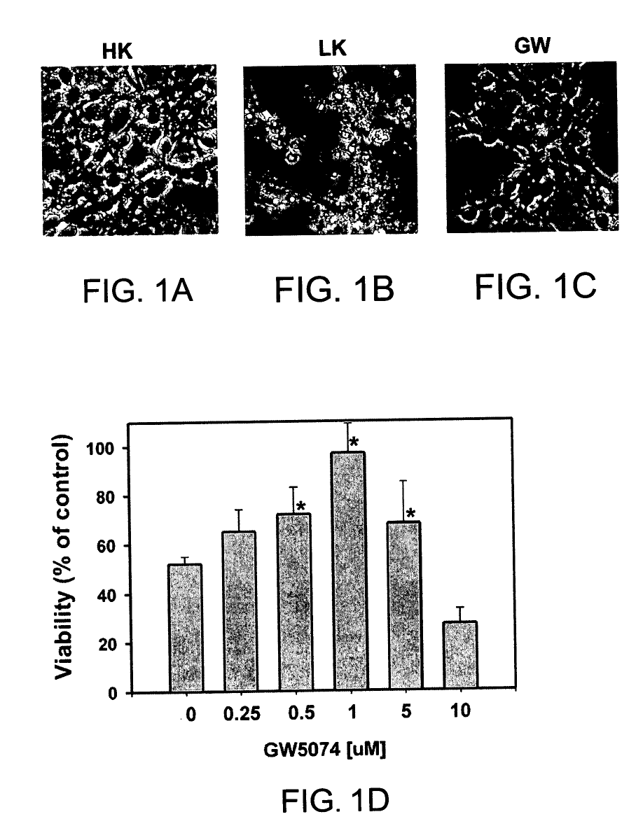

FIGS. 1A-D shows the neuroprotective effect of the C-Raf inhibitor, {5-Iodo-3-[(3,5-dibromo-4-hydroxyphenyl) methylene]-2-indolinone} (“GW5074”), according to a preferred embodiment of the invention. Cultured cerebellar granule neurons undergo apoptosis when switched from HK to medium containing LK (D'Mello et al., 1993). However, as shown in FIGS. 1A-1D, treatment with GW5074 prevents LK-induced apoptosis in these cultures. FIGS. 1A-1C provide phase contrast micrographs showing the morphological appearance of cerebellar neuronal cultures treated with high potassium (HK)(FIG. 1A), low potassium (LK) (FIG. 1B), or LK+1 uM GW5074 for 24 hours (FIG. 1C). Cerebellar neurons exposed to low potassium conditions but treated with the C-Raf inhibitor, GW5074, survive as well as neurons exposed to normally high potassium conditions. FIG. 1D quantifies the anti-apoptotic effect of GW5074. Neuronal cultures were switched to medium containing LK, or LK medium containing different doses of GW5074. Cell viability was quantified 24 hours later. Control cultures received HK medium. (Results shown are from three separate experiments. *p<0.001 mean value +SD compared with viability of culture receiving LK medium with no GW5074). Protection against LK-induced apoptosis is maximal at 1 uM. Neuroprotection by GW5074 is also observed at concentrations at 5 uM and 10 uM.

As shown in Table 1, GW5074 is a potent and specific inhibitor of C-Raf in vitro. GW5074 is a potent inhibitor of C-Raf with no effect on the activities of cdk1, cdk2, c-src, p38 MAP kinase, VEGFR2, and c-fms. (Lackey et al., 2000). GW5074 potently inhibits C-Raf (TABLE 1). However, although clearly a potent inhibitor of C-Raf, the possibility that GW5074 inhibits other kinases that may have proapoptotic effects could not be excluded. A number of reports have implicated JNKs in the promotion of neuronal apoptosis both in vivo and in cell culture systems (Coffey et al., 2002). Although all three JNK proteins are expressed in neurons, the phosphorylation of c-jun is believed to be mediated by JNK2 or JNK3 (Bruckner et al., 2001; Bruckner and Estus, 2002; Coffey et al., 2002). As shown in TABLE 1, GW5074 has no direct effect on the activity on any of the three JNK proteins. In apoptotic neurons, JNK activation is mediated primarily by MKK7 (Eilers et al., 1998; Trotter et al., 2002). As shown in TABLE 1, GW5074 has no effect of MKK7 activity. Additionally, GW5074 did not affect MKK6, a kinase that activates p38 MAP kinase, another stress-activated MAP kinase implicated in neuronal apoptosis (TABLE 1). The activity of each kinases was measured in vitro in the presence of 1 uM GW5074 and in all cases ATP was 10 uM. (Kinase activity is expressed as a percentage of that in control (without GW5074) and results are expressed as mean+/−standard deviation).

TABLE 1

| KINASES | % ACTIVITY |
|---|---|
| C-Raf | 3 ± 0 |
| JNK1 | 94 ± 3 |
| JNK2 | 91 ± 4 |
| JNK3 | 89 ± 9 |
| MEK1 | 94 ± 4 |
| MKK6 | 91 ± 4 |
| MKK7 | 104 ± 8 |
| CDK1/cyclinB | 90 ± 12 |
| CDK2/cyclinE | 100 ± 5 |
| CDK2/cyclinA | 99 ± 0 |
| CDK5/p35 | 94 ± 2 |
| CDK6/cyclinD3 | 98 ± 1 |
| GSK3β | 81 ± 6 |

Several lines of evidence indicate that abortive reentry into the cell cycle by activation of cell cycle components is responsible for apoptosis in granule neurons and other neuronal types (Park et al., 1997a, 1997b, 1998a, 1998b; Padmanabhan et al., 1999). It was thus possible that the neuroprotective effect of GW5074 was mediated by cdk inhibition. As shown in TABLE 1, however, GW5074 had no effect on the activity of any of the cdks that were examined.

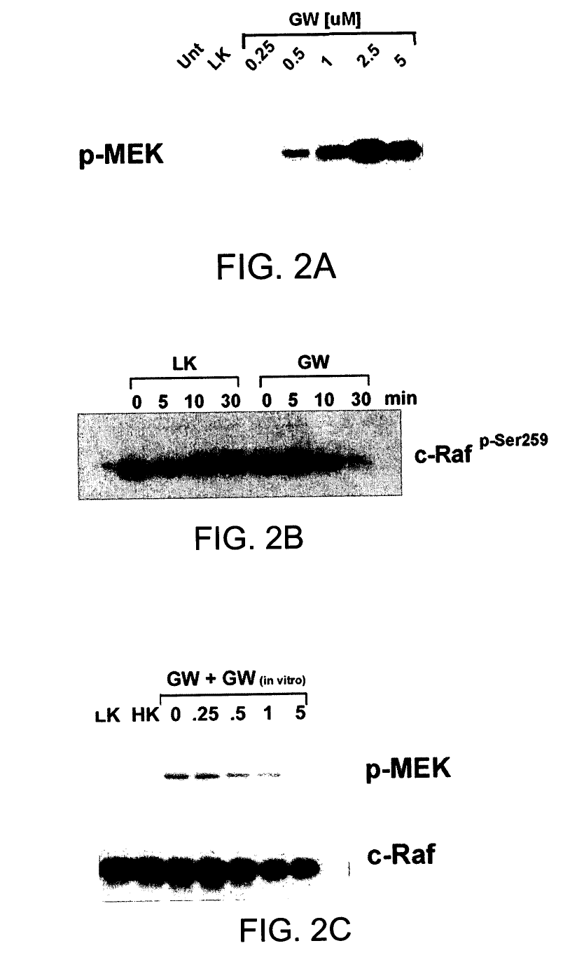

FIGS. 2A-2C shows that a highly neuroprotective C-Raf inhibitor, GW5074, nonetheless permits activating modifications to accumulate in intact neurons as indicated by an in vitro kinase assay using an MEK substrate. Also shown is the extent of Ser29 phosphorylation of C-Raf indicating strong inhibition in neuronal cultures and in vitro. To examine whether C-Raf was inhibited by GW5074 in granule neurons, cultured neurons were treated with GW5074 and the lysates used to immunoprecipitate C-Raf. The immunoprecipitated C-Raf was used in in vitro kinase assays using GST-MEK as substrate and the reaction products subjected to Western analysis. Phosphorylation of MEK was detected using a phospho-MEK antibody. Neurons were either untreated or switched to LK medium containing no additives (LK) or doses of GW5074 (GW) ranging from 0.25-5 uM. After 1 hour of treatment, the cultures were lysed, C-Raf immunoprecipitated from the lysate and used in an in vitro kinase assay with MEK as substrate. The reaction mixture was subjected to Western analysis using a phospho-MEK antibody as probe. As shown in FIG. 2A, C-Raf activity is barely detectable in cells. This low level of activity remained unchanged in cultures switched to LK medium. Also shown in FIG. 2A, the activity of C-Raf immunoprecipitated from cultures treated with GW5074 in LK medium displayed a marked induction (FIG. 2A).

FIG. 2B shows that C-Raf is inhibited by phosphorylation at Ser259 and activation of C-Raf requires dephosphorylation of this site (Dhillon and Kolch, 2002; Hindley and Kolch, 2002). Neuronal cultures were switched to LK medium containing no additives (LK) or 1 uM GW5074 for 0, 5, 10, and 30 min. The cultures were lysed and the lysate subjected to Western blot analysis using a phopho-Ser259-specific antibody. As shown FIG. 2B, when neurons are switched to LK medium, there is a sustained increase in phosphorylation of C-Raf at Ser259 which can be detected within 10 min. A rapid increase in Ser259 phosphorylation is also seen following treatment with GW5074.

To study the effect of GW5074 on C-Raf activity further, C-Raf was immunoprecipitated from granule neurons treated with GW5074 and assayed its activity in vitro in the absence or presence of GW5074. As shown in FIG. 2C, while C-Raf immunoprecipitated from GW5074-treated cultures has higher activity relative to control cultures (without GW5074), addition of GW5074 to the immunoprecipitated enzyme in vitro inhibits its activity confirming that GW5074 inhibits C-Raf and does so at concentrations which are substantially lower than 1 uM, a dose at which the drug is neuroprotective. Inhibition of C-Raf by compounds such as GW5074 could allow activating modifications (such as Ser259 dephosphorylation) to accumulate which may stimulate other kinases through compensatory mechanisms.

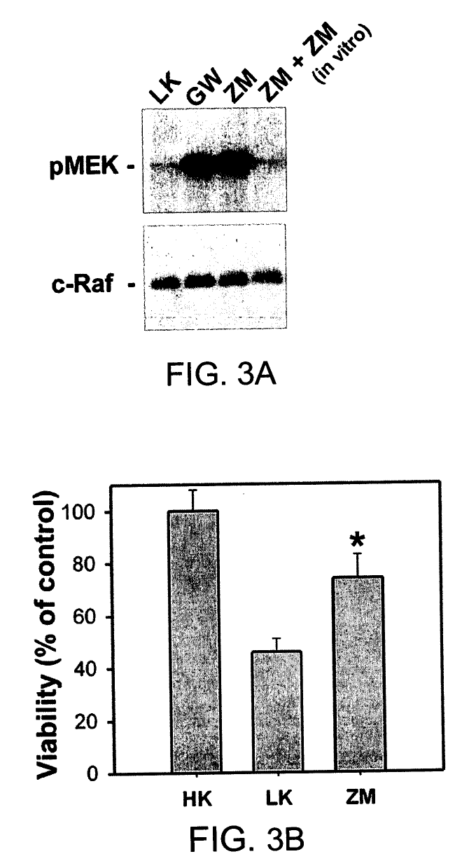

FIGS. 3A-3B shows the neuroprotective effect of a C-Raf inhibitor, ZM33672, according to another embodiment of the invention. ZM336372 is a pharmacological inhibitor of C-Raf; it has the chemical name N-[5-(3-Dimethylaminobenzamido)-2-methylphenyl]-4-hydroxybenzamide and is obtained from CALBIOCHEM, Catalog No. 692000. (Hall-Jackson et al., 1999). While GW5074 inhibits C-Raf with an IC₅₀ of 9 nM, ZM336372 is somewhat less potent (IC₅₀=70 nM; Hall-Jackson et al., 1999). Neuronal cultures were switched to HK, LK, or LK medium containing 100 uM ZM336372. As shown in FIG. 3A, immunoprecipitated C-Raf from neuronal cultures treated with ZM336372 has elevated activity, which is inhibited in vitro by ZM336372. To assess the efficacy of ZM336372, neuronal cultures were treated for 1 hour with LK, or LK containing 1 uM GW5074 (GW) or 50 uM ZM336372 (ZM). Lysates from the cultures were used in a kinase reaction with MEK as substrate. In one assay from GW5074-treated cultures, 10 uM ZM336372 was added to the kinase reaction mixture. Phosphorylation of MEK was detected by Western blot analysis using an phospho-MEK antibody and the same blot was reprobed with a C-Raf antibody. As shown in FIG. 3B, ZM336372 inhibits LK-mediated apoptosis. The ability of an independent and structurally distinct pharmacological C-Raf inhibitor to promote neuronal survival suggests that the neuroprotective effect of GW5074 is either directly or indirectly due to its action on C-Raf.

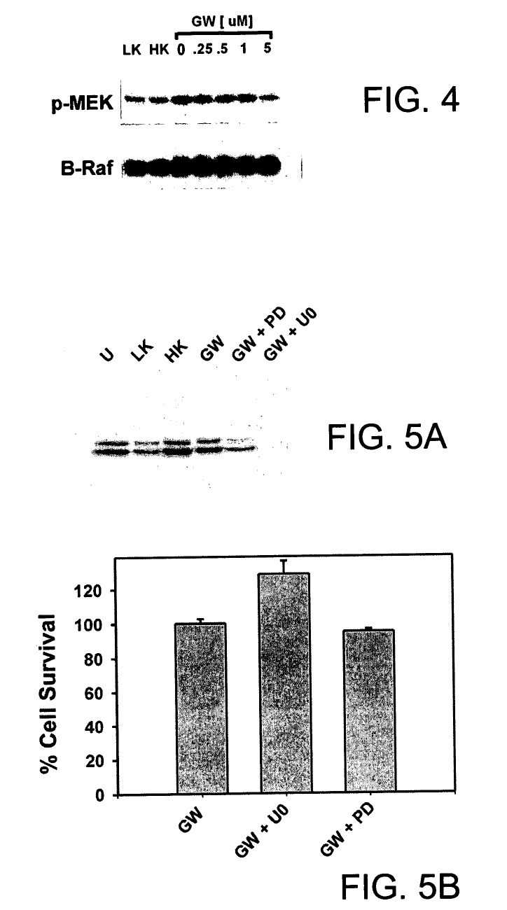

FIG. 4 shows that GW5074 activates B-Raf and ERK . Activation of all three Raf proteins results in activation of MEK and ERK. Lysates from neuronal cultures treated with LK or LK plus GW5074 (GW) for 1 or 3 hours were subjected to Western blotting using an antibody specific for phosphorylated ERK 1/2. As shown in FIG. 4, despite its inhibition of C-Raf, treatment of neuronal cultures with GW5074 leads to an increase in the phosphorylation of ERK 1/2 suggesting that GW5074 treatment caused the activation of either A-Raf or B-Raf.

Since B-Raf is highly expressed in the nervous system and since B-Raf rather than C-Raf is the major stimulator of ERK both in vivo and in vitro, the effect of GW5074 on B-Raf was examined. Neuronal cultures were switched to medium containing HK or LK medium containing no additives (LK) or 1 uM GW5074 (GW). After 1 hour of treatment, cultures were lysed, C-Raf immunoprecipitated from the lysate and used in an in vitro kinase assay with MEK as substrate. Immunoprecipitates from the GW5074-treated culture were incubated with different doses of GW5074 ranging from 0.25 to 5 uM during the in vitro kinase reaction, then subjected to Western analysis using a phospho-MEK antibody as probe. The same blot was reprobed with an antibody to B-Raf. To assess in vitro kinases activity of B-Raf, neuronal cultures were switched to medium containing HK or LK medium containing no additives (LK) or 1 uM GW5074 (GW). After 1 hour of treatment, cultures were lysed, C-Raf immunoprecipitated from the lysate and used in an in vitro kinase assay with MEK as substrate. Aliquots of the immunoprecipitate from the GW5074-treated culture were incubated with different doses of GW5074 ranging from 0.25 to 5 uM during the in vitro kinase reaction. The reaction mixture was subjected to Western analysis using a phospho-MEK antibody as probe. The same blot was reprobed with an antibody to B-Raf. As shown in FIG. 4, cerebellar granule neurons have relatively high B-Raf activity and is elevated further by treatment with GW5074. Consequently, it is likely that inhibition of C-Raf leads to the activation of B-Raf. Neuroprotection by C-Raf inhibition therefore potentially activates a novel neuroprotective pathway involving B-Raf.

Moreover, further experiments show that B-Raf activation is a critical event in the neuroprotective effect of GW5074. A dominant-negative N-Raf expression vector, pSRa GST B-Raf S728A (full length human B-Raf with Serine to Alanine mutation at position 728) was gifted to us by Dr. Angus M. MacNicol, University of Arkansas for Medical Sciences, Little Rock, Ark. Neuronal cultures were transfected with CMV-LacZ or the dominant-negative B-Raf construct using the calcium phosphate method 5 days after plating of the cultures. The next day, the neurons were switched to LK medium containing 1 uM GW5074. The proportion of transfected neurons that were apoptotic was quantified 24 hours later. Inhibition of B-Raf in GW5074-treated cultures by infection using plasmids expressing a dominant-negative form of B-Raf leads to the death of 80% of the infected neurons (data not shown). In contrast, overexpression of LacZ (as a control) leads to the death of less than 10% of the neurons. In view of the involvement of B-Raf in the neuroprotective action of C-Raf inhibitors, therefore, activators of B-Raf could be used to treat neurodegenerative conditions.

FIGS. 5A-5B shows that GW5074-mediated neuroprotection is MEK-ERK independent. Survival of cerebellar granule neurons can be maintained by BDNF and this effect of BDNF is mediated by the Raf-MEK-ERK signaling pathway (Bonni et al., 1999). The Raf-MEK-ERK pathway is also involved in promoting survival of other neuronal and normeuronal cell types. A potent blocker of the Raf-MEK-ERK pathway is PD98059 (Alessi et al., 1995). In paradigms in which the Raf-MEK-ERK pathway mediates neuronal survival, such as BDNF-treated cerebellar granule neurons, the presence of PD98053 blocks survival (Bonni et al., 1999). To determine whether GW5074-mediated neuroprotection is MEK-ERK independent, neuronal cultures maintained in serum and high K+ were either untreated or switched for 1 hour to HK, LK, or LK medium containing 1 uM GW5074 in the absence (GW) or presence of 40 uM PD98059 (GW+PD) or 10 uM U0126 (GW+UO). Lysates from the cultures were subjected to Western blotting using an antibody specific for phospho-ERK. As shown in FIG. 5A. treatment with PD98059 blocks the stimulation of ERK by GW5074. This, however, had no effect on the neuroprotective effect of GW5074 (FIG. 5B) as cell viability remained the same after cultures subjected to LK conditions. U0126, a structurally independent MEK inhibitor, which potently inhibits both MEK1 and MEK2 (Duncia et al., 1998; Favata et al., 1998 and FIG. 5A) also failed to reduce survival by GW5074 (FIG. 5 A and B). These results show that neuroprotection by GW5074 is MEK-ERK independent.

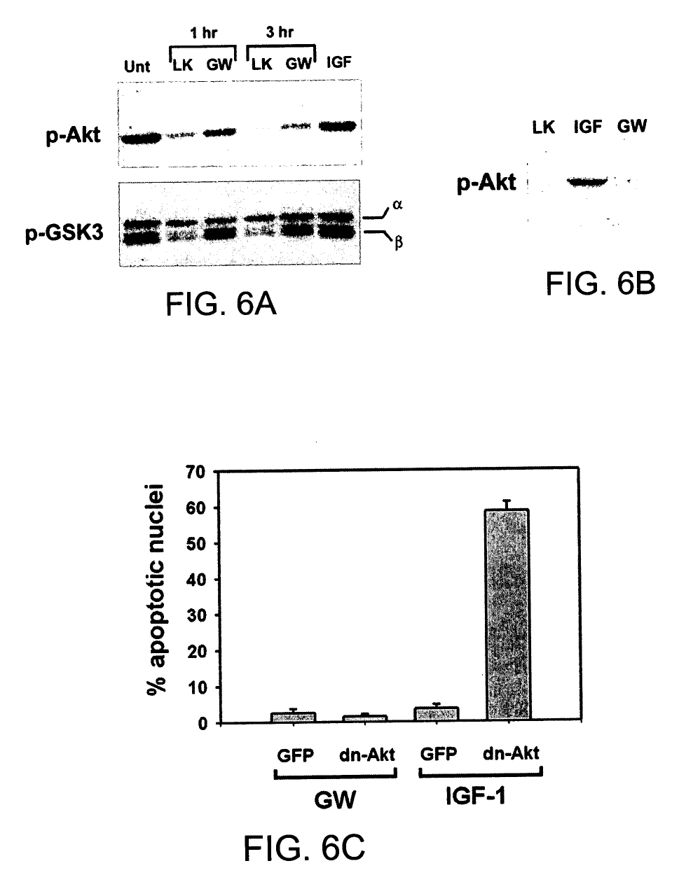

FIGS. 6A-6C shows that GW5074 maintains Akt activity but acts through an Akt-independent mechanism. The best-studied anti-apoptotic pathway in neurons is the PI-3 kinase—Akt signaling pathway (D'Mello et al., 1997; Dudek et al., 1997; Miller et al, 1997a). IGF-1, a potent survival-promotic factor for granule neurons mediates its effect by activating the PI-3 kinase-Akt pathway (Datta et al., 1997; Dudek et al., 1997; D'Mello et al., 1998). Since the Raf-MEK-ERK pathway was not necessary for the neuroprotective action of GW5074, the possibility that GW5074 exerted its protective effect by engaging this pathway was examined. Switching cerebellar granule neurons from serum-containing HK medium in which they are cultured and allowed to mature, to serum-free LK or HK medium, causes a rapid downregulation of Akt phosphorylation and activity, which can be prevented by IGF-1 (Kumari et al., 2001). To assess the role of Akt in GW5074-mediated neuroprotection, Lysates from neurons treated for 1 hour and 3 hours with LK or LK medium containing 1 uM GW5074 (GW) were subjected to Western blotting using an antibody against phospho-Akt (Ser473). The same blot was reprobed with an antibody against phospho-Gskβ. Also loaded on the gel were lysates from untreated cultures (maintained in medium containing serum and high K+) and a culture treated for 1 hour with 25 ng/ml IGF-1. As also shown in FIG. 6A, GW5074 delays the downregulation of Akt phosphorylation observed after LK treatment. GSK3βis a proapoptotic molecule that is activated during apoptosis in many neuronal and normeuronal systems. Under survival promoting conditions GSK3β is kept inactivated by phosphorylation, a modification that can be induced by Akt. As shown in FIG. 6A, GW5074 prevents the activation of GSK3, that occurs after the switch to LK medium. Another proapoptotic molecule that is phosphorylated and inactivated by Akt is the transcription factor, Forkhead (Linseman et al., 2002). GW5074 reduces the dephosphorylation of Forhkead that is observed in LK (data not shown). Switching of neurons to LK medium leads to a rapid downregulation of Akt activity within 2 hours. Addition of GW5074 to such cultures does not lead to Akt activation. In contrast, addition of IGF-1 causes a robust increase in Akt phosphorylation (FIG. 6B). Thus, while capable of temporarily maintaining the activity of Akt in LK, treatment with GW5074 cannot activate Akt de novo.

To determine if Akt activity was necessary for the neuroprotective action of GW5074, neurons were treated with GW5074 after infecting them with an adenoviral vector expressing a dominant-negative form of Akt. Five day old neuronal cultures were infected with adenoviral vectors expressing either GFP or hemagluttinin (HA)-tagged dominant-negative Akt. The next day the cultures were switched to LK medium containing 1 uM GW5074 (GW) or 25 ng/ml IGF-1 (IGF). Infected neurons were detected by positive staining for GFP or HA by immunocytochemistry. The proportion of apoptotic cells (condensed or fragmented nuclei) as a percentage of total infected neurons was quantified following DAPI-staining. As shown in FIG. 6C, while blockade of Akt activity using this approach reduces the survival-promoting effect of IGF-1, it had no effect on the ability of GW5074 to maintain neuronal survival. This result indicates that although maintaining Akt activity in LK, the neuroprotective action of GW5074 is mediated by an Akt-independent mechanism.

FIGS. 7A-7C shows that GW5074-mediated neuroprotection involves Ras, NFKB and c-jun. Activation of C-Raf and B-Raf is often mediated by Ras. Blockade of C-Raf signaling by GW5074 could lead to an accumulation of activated Ras, which could lead to the stimulation of an alternative, antiapoptotic pathway. While known to exert antiapoptotic effects by a PI-3 kinase-Akt dependent pathway, Ras has also been shown to provide an antiapoptotic signal through an Akt-independent mechanism involving downregulation of JNK and p38 activity (Wolfman et al., 2002). To determine if Ras was necessary for GW5074-mediated neuroprotection, S-trans, trans-farnesylthiosalicylic acid (FTS) was used. FTS is a cell permeable Ras antagonist that dislodges Ras from its membrane-anchoring sites leading to its degradation and thus causing a decrease in total cellular Ras (Jansen et al., 1999; Weisz et al., 1999). In assessing the role of Ras, neuronal cultures were switched to medium containing HK or to LK medium containing 1 uM GW5074 in the absence (GW) or presence of 10 uM FTS (GW+FTS) and viability was quantified 24 hour later and expressed as percentage of viability in HK. As shown in FIG. 7A, treatment with FTS blocks the neuroprotective effect of GW5074.

Another molecule known to be important for neuronal survival is NF-κB. Although the precise mechanism has not been elucidated, the Raf is known to activate NF-κB in normeuronal cells via a MEK-ERK-independent mechanism (Foo and Nolan, 1999; Pearson et al., 2000). The effect of GW5074 treatment on the DNA-binding activity of NF-κB was examined. This was assessed by switching neuronal cultures to medium containing HK or to LK medium containing 1 uM GW5074 in the absence (GW) or presence of 10 uM FTS (GW+FTS). Viability was quantified 24 hours later and expressed as percentage of viability in HK. As shown in FIG. 7B, treatment of cerebellar neuron cultures with LK leads to a downregulation of NF-κB activity. As shown in FIG. 7B, this downregulation of NF-κB binding activity is prevented by GW5074.

One potential mediator of MEK-ERK-independent B-Raf signaling is NF-κB. Whether treatment NF-κB was required for the neuroprotective effect of GW5074 was examined. To do so, nuclear extracts from cultures treated for 6 hours with HK, LK, or LK medium containing 1 uM GW5074 (LK+GW) were used in gel mobility shift assays with radioactively-labeled oligonucleotide probes containing the NF-κB binding site or the SPI binding site. As shown in FIG. 7C, treatment with SN-50 (Lin et al. 1995), a synthetic cell-permeable peptide that has been demonstrated to potently and specifically inhibit NF-κB activity, blocks the ability of GW5074 to inhibit LK-induced apoptosis indicating that the neuroprotective effect of GW5074 is NF-κB-dependent.

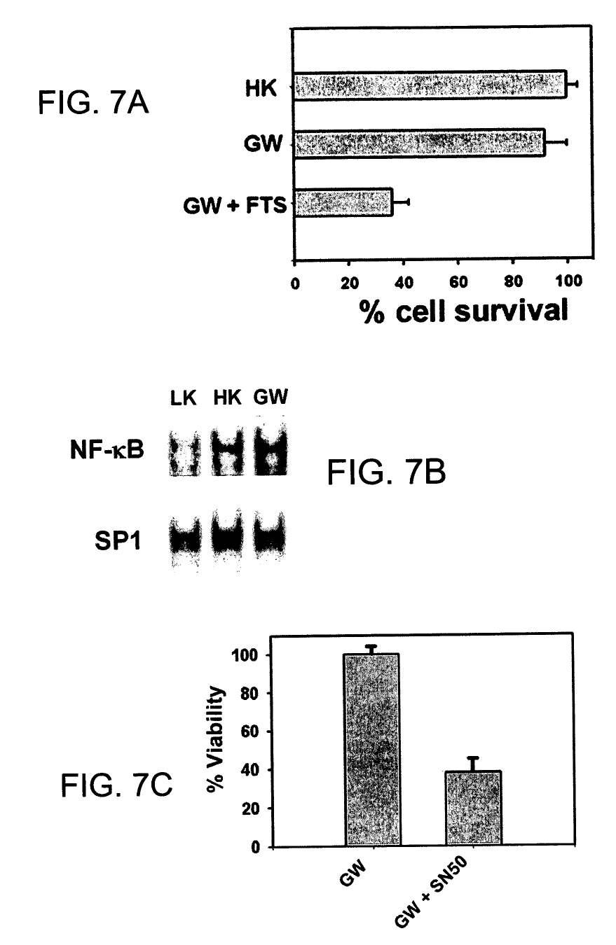

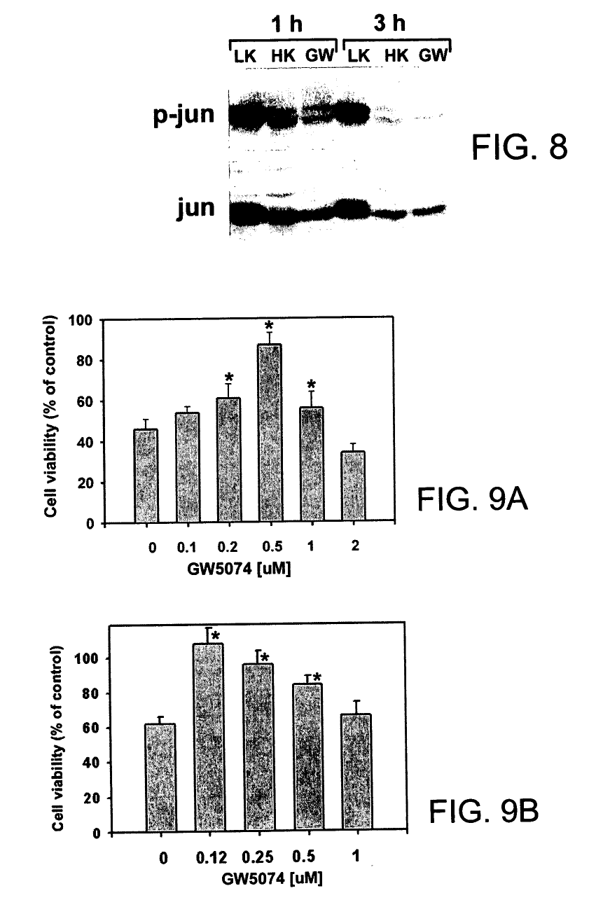

FIG. 8 shows that GW5074 inhibits apoptosis-associated induction of c-jun. Phosphorylation of c-jun is necessary for neuronal apoptosis in a variety of paradigms including LK-induced death of granule neurons (Estus et al., 1994; Ham et al., 1995; Watson et al. 1998).

In granule neurons, phosphorylation of c-jun occurs within 1 hour of LK treatment (Ham et al., 1995; Watson et al. 1998). To assess the role of c-jun, lysates from neurons treated for 1 hour and 3 hours with HK, LK or LK medium containing 1 uM GW5074 (GW) were subjected to Western blotting using an antibody against phospho-c-jun (Ser63). The same blot was reprobed with an antibody against total c-jun (lower panel). As shown in FIG. 8, GW5074 treatment prevents LK-induced c-jun phosphorylation and its increased synthesis.

FIGS. 9A-9B shows that GW5074 inhibits cell death caused by neurotoxins in granule cells and other neuronal types. The ability of different doses of GW5074 to protect against apoptosis induced by various stimuli was tested in cultured cerebellar granule neurons (FIGS. 9A and B). Methyl-4-phenyl-1,2,3,6-tetrathydropyridine (MPTP) is a neurotoxin that causes degeneration of nigrostratial dopaminergic neurons in humans and some experimental animals resulting in a Parkinson's like pathology. The neurotoxic effects of MPTP are mediated through its oxidation by astrocytes to the neurotoxic species 1-methyl-4-phenylpyridinium (MPP+) that is taken up actively by dopaminergic neurons through the dopamine transporter. Direct treatment of dopaminergic neurons in culture with MPP+recapitulates their degeneration seen in vivo. In culture, MPP+ is also toxic to cerebellar granule neurons (Gonzalez-Polo et al., 2001). Another neurotoxic agent that causes selective loss of cerebellar granule neurons in vivo and which induces apoptosis in cultured cerebellar granule neurons is methylmercury (Kunimoto, 1994). As a step towards determining whether GW5074 is protective against other neurotoxic stimuli its ability to prevent cell death in granule neuron cultures treated with MPP+ or methylmercury was examined. To do so, cerebellar granule neurons were treated with HK medium or HK medium containing 200 uM MPP+ (FIG. 9A) or 0.5 uM methylmercury (FIG. 9B) in the presence of different concentrations of GW5074 (GW). As shown in FIG. 9A, GW5074 reduced MPP⁺-induced cell death. Similarly, as shown in FIG. 9B, GW5074 also reduced methylmercury-induced cell death.

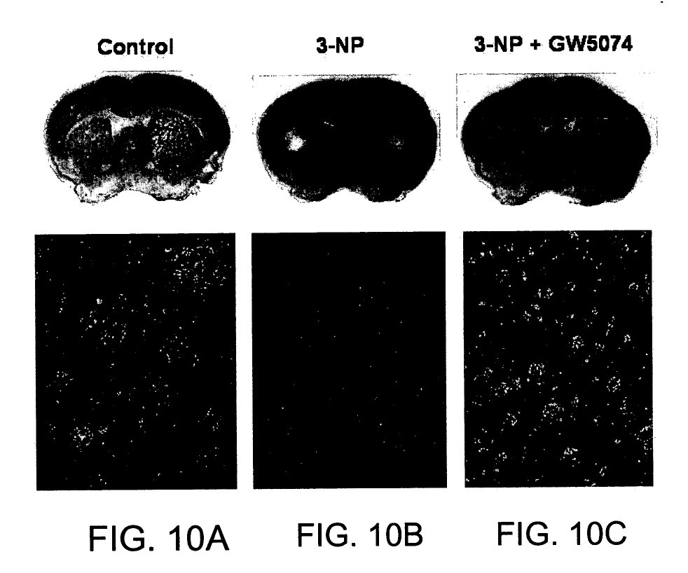

FIGS. 10A-10C shows that GW5074 is protective in an in vivo experimental model of Huntington's disease. 3-Nitropropionic acid (3-NP) administration in rodents and nonhuman primates has served as a useful experimental model for HD (reviewed in Brouillet et al., 1999). 3-NP is an irreversible inhibitor of succinate dehydrogenase (SDH; Complex II), which causes prolonged mitochondrial energy impairment and replicates most of the clinical and pathophysiological hallmarks of HD including selective striatal degeneration, spontaneous choreiform and dystonic movements (Brouillet et al., 1999). Whether GW5074 could protect against 3-NP-induced neurodegeneration was tested. FIG. 10A shows the Control. As shown in FIG. 10B, mice administered with 3-NP display extensive bilateral striatal lesions. FIG. 10C shows this degeneration is completely prevented by GW5074 when administered at a concentration of 5 mg/kg body weight. A similar protection was also observed when GW5074 was administered at 25 mg/kg.

### Materials and Methods

Materials.

Unless specified otherwise, all chemicals were purchased from Sigma Chemicals (St. Louis, Mo.). All antibodies used were purchased from Cell Signaling, Inc. (Beverly, Mass.) unless specified otherwise. PD98059, S-trans, trans-Farnesylthiosalicyclic Acid (FTS), and SN-50 were purchased from Calbiochem (La Jolla, Calif.), U0126 was purchased from Cell Signaling Technology (Beverly, Mass.), and ZM336372 was purchased from Tocris (Ellisville, Mo.). The Adenoviral expression vector encoding dominant-negative Akt was gifted by Thomas Franke, Columbia University, New York, N.Y.). The expression plasmid encoding a kinase dead GST-MEK1 was a kind gift of Melanie Cobb.

Cell Culture and Treatments.

Granule neuron cultures were obtained from dissociated cerebella of 7-8 day old rats as described previously (D'Mello et al., 1993). Cells were plated in Basal Eagle's Medium with Earles salts (BME) supplemented with 10% fetal bovine serum (FBS), 25 mM KCl, 2 mM glutamine (Gibco-BRL), and 100 ug/ml gentamycin on dishes coated with poly-L-lys ine in 24-well dishes at a density 1.0×10⁶ cells/well, 1.2×10⁷ cells/60 mm dish, or 3.0×10⁷ cells/100 mm dish. Cytosine arabinofuranoside (10 uM) was added to the culture medium 18-22 hours after plating to prevent replication of non-neuronal cells. Cultures were maintained for 6-7 days prior to experimental treatments. For this, the cells were rinsed once and then maintained in low K⁺ medium (serum-free BME medium, 5 mM KCl) or high K⁺ medium (serum-free BME medium, supplemented with 20 mM KCl). When used to treat cultures, GW5074 was added at the time when cells were switched to LK medium. Treatment of cultures with pharmacological inhibitors was initiated 15 min prior to rinsing and was maintained through the subsequent incubation in LK or HK medium unless specified otherwise. For MPP+ and methylmercury treatments, 7-8 day old cultures were switched to HK medium containing 200 uM MPP+ or 0.5 uM methylmercury. Viability was assayed 24 hours later.

Neuronal Survival

Neuronal survival was quantified by the MTT assay as previously described (Koulich et al., 2001). Briefly, the tetrazolium salt MTT [3-(4,5-dimethyl-2-thiazolyl)-2,5-diphenyl-2H tetrazolium bromide] was added to the cultures at a final concentration of 1 mg/ml, and incubation of the culture was continued in the CO₂ incubator for a further 30 min at 37° C. The assay was stopped by adding lysis buffer [20% SDS in 50% N,N-dimethyl formamide, pH 4.7]. The absorbance was measured specrophotometrically at 570 nm after an overnight incubation at room temperature. The absorbance of a well without cells was used as background and subtracted. Results obtained using the MTT assays were confirmed using the fluorescein-diacetate method for quantification of cell viability, as previously described (D'Mello et al., 1993). Data are presented as mean+/−standard deviation. Statistical analysis was performed using ANOVA and Student-Neuman-Keuls' test.

Western Blotting.

For whole-cell lysates, the culture medium was discarded, the neurons washed twice with ice-cold phosphate-buffered saline (PBS), and lysed in lysis buffer (1% Triton, 20 mM Tris-HCl (pH 7.5), 150 mM NaCl, 1 mM Na₂EDTA, 1 mM EGTA, 2.5 mM sodium pyrophosphate, 1 mM beta-glycerophosphate, 1 mM Na₃VO₄, 1 μg/ml leupeptin and 1× protease inhibitor mixture). Protein concentrations were measured using a Bradford protein assay kit (Bio-Rad), and equivalent amounts of protein were mixed with 6×SDS-PAGE sample buffer. Following heating at 95° C. for 5 min, proteins were subjected to SDS-polyacrylamide gel electrophoresis and transferred electrophoretically to polyvinylidene difluoride membrane (PVDF; Bio Rad). After staining with Ponceau S to verify uniformity of protein loads/transfer, the membranes were analyzed for immunoreactivity. Incubation with primary antibodies was performed overnight at 4° C. and with secondary antibodies for 1 h at room temperature. Immunoreactivity was developed by enhanced chemiluminescence (ECL; Amersham) and visualized by autoradiography. All antibodies were from Cell Signaling.

Immunoprecipitation.

After treatment, cultures of 7-8-day old neurons were washed twice with ice-cold PBS and lysed in ice cold lysis buffer. The lysates were centrifuged for 10 min at 10,000 rpm at 4° C. Protein concentrations of the supernatant were measured using a Bradford protein assay kit (Bio-Rad), and equivalent amounts of protein were incubated overnight with primary antibody (1.0-2 ug) and then for 2 h with 20 ul Protein A/G PLUS-Agarose (Santa Cruz Biotech.) Immunoprecipitates were collected by centrifugation at 2,500 rpm for 5 min at 4° C. and washed three times with lysis buffer, and pellets resuspended in electrophoresis sample buffer (187.5 mM Tris-HCl (pH 6.8 at 25° C.), 6% SDS, 30% glycerol, 150 mM DTT, 0.03% bromophenol blue), boiled for 4 min and subjected to SDS-polyacrylamide gel electrophoresis.

In Vitro Kinase Assays:

In general, in vitro kinase assays were performed using purified kinase and synthetic substrates under standard conditions using the Kinase Profiling service of Upstate Biotechnology. Briefly, for each assay 5-10 mU of purified kinase was used. For GSK3p, cdk1, cdk2, cdk3, cdk5, the kinase was incubated with 1 uM GW5074 in a buffer containing 8 mM MOPS, pH 7.2, 0.2 mM EDTA, 10 mM Mg Acetate and [γ-³³P-ATP] for 40 min at room temperature. Kinase activity was quantified by measuring ³³P incorporation by spotting an aliquot on P30 filters, washing in 50 mM phosphoric acid and scintillation counting. The buffer composition for C-Raf, JNK1, JNK2, JNK3, MEK1, MKK6, MKK7 was 50 mM Tris pH7.5, 0.1 mM EGTA, 10 mM Mg acetate and [γ-³³P-ATP]. The peptide substrates used were as follows: For C-Raf, 0.66 mg/ml MBP; for cdks, 0.1 mg/ml histone H1; for JNKs, 3 uM ATF2; for MEK1, 1 uM MAPK2; for MKK6, 1 uM of SAPK2a and for MKK7, 2 uM JNK1α.

Activities of endogenous C-Raf and B-Raf activity was assayed by measuring the ability of kinase immunoprecipitated from neuronal lysates to phosphorylate a kinase-dead recombinant GST-MEK1 substrate. Following immunoprecipitation and multiple washes with lysis buffer, lysis buffer supplemented with 350 mM NaCl, and kinase buffer (25 mM HEPES pH 7.4 and 10 mM MgCl₂), in vitro kinase assays are performed on the immune complexes using purified recombinant GST-MEK1 K97M protein as a substrate in kinase buffer supplemented with 85 μM ATP for 35 minutes at 30° C. Reactions are stopped by the addition of 3×SDS sample buffer and boiled for five minutes. Proteins are resolved by SDS-PAGE and subjected to Western blotting. The level of phosphorylated MEK is detected by a phospho-MEK antibody.

Overexpression Using Adenoviral Vectors:

The hemagglutinin-tagged dominant-negative Akt (Ad-dnAkt) consists of two mutations where the phosphorylation sites at Thr308 and Ser473 are mutated to yield a phosphorylation-deficient inactive protein. DnAkt and control recombinant adenovirus expressing green fluorescent protein (Ad-GFP) are propagated in HEK293T cells and purified by cesium chloride density gradient centrifugation. After quantification of titer, virus at an MOI of 10 are used to infect granule neuron cultures 5 days after plating by direct addition to the medium. Treatments are performed 24 hours after addition of virus.

Gel Electrophoresis Mobility Shift Assay:

Nuclei from neuronal cultures were resuspended in buffer containing 20 mM HEPES pH 7.6, 50 mM KCl, 300 mM NaCl, 0.1 mM EDTA, 1 mM DTT, 0.1 mM PMSF, 10% glycerol, 1× protease inhibitor cocktail and extracted on ice for 30 min, followed by micro-centrifugation at 14,000 rpm for 10 min. The supernatants were collected as nuclear extracts. Concentrations of these nuclear extracts were determined by the Bradford method using reagents from Bio-Rad. Ten μg of each nuclear extract sample was incubated with 0.1 pmol of ³²P-labeled double-stranded KB binding oligonucleotide (5′-GCTGGGGACTTTC-3′) identified as SEQ ID NO:1, or SP1 binding oligonucleotide (5′-ATTCGATCGGGGCGGGGCGAGC-3′) identified as SEQ ID NO:2, in buffer containing 1 μg of poly (d1-dC), 1 μg of BSA, 10 mM HEPES pH7.6, 0.5 mM DTT, 0.1 mM EDTA, 60 mM KCl, 0.2 mM PMSF, 5 mM MgCl2, and 12% glycerol at room temperature for 30 min. Samples were analyzed by 5% native polyacrylamide gel electrophoresis (PAGE) followed by autoradiography.

3-Nitropropionic Acid Treatment and Experimental Design:

Mice were purchased from Charles River (Wilmington, Mass.). 3-NP was dissolved in water and the solution brought to pH 7.4 with sodium hydroxide. 3NP was administered to 8-week old B6CBA male mice in ten intraperitoneal injections (50 mg/kg twice a day for 5 days). GW5074 was also administered intraperitoneally at doses of 0.5 to 10 mg/kg once a day each day 3-NP was administered. Injected of GW5074 was performed 30 minutes to 1 hour before 3NP administration. Control animals received saline injections. On the day following the 5 days of injection, mice were deeply anesthetized, intracardially perfused, brains removed, post fixed in 4% paraformaldehyde and cryoprotected. Coronal sections were cut on a cryostat at 50 micron and stained for Nissl substance (cresyl violet).

Various basics of the invention have been explained herein. The various techniques and devices disclosed represent a portion of that which those skilled in the art would readily understand from the teachings of this application. Details for the implementation thereof can be added by those with ordinary skill in the art. The accompanying figures may contain additional information not specifically discussed in the text and such information may be described without adding new subject matter. Additionally, various combinations and permutations of all elements or applications can be created and presented. All can be done to optimize performance in a specific application.

The various steps described herein can be combined with other steps and they can occur in a variety of sequences unless otherwise specifically limited. These various steps can be interlineated with the stated steps, and the stated steps can be split into multiple steps. Unless the context requires otherwise, the word “comprise” or variations such as “comprises” or “comprising”, should be understood to imply the inclusion of a stated element or step or group of elements or steps but not the exclusion of any other element or step or group of elements or steps.

Further, any references mentioned in the application for this patent as well as all references listed in any list of references filed with the application are hereby incorporated by reference. However, to the extent statements might be considered inconsistent with the patenting of this invention, such statements are expressly not to be considered as made by the applicant(s).

### REFERENCES

Alessi D R, Cuenda A, Cohen P, Dudley D T, Saltiel A R. (1995) PD 098059 is a specific inhibitor of the activation of mitogen-activated protein kinase kinase in vitro and in vivo. J Biol. Chem. 270, 27489-27494.
Anderson C N, Tolkovsky A M. (1999) A role for MAPK/ERK in sympathetic neuron survival: protection against a p53-dependent, JNK-independent induction of apoptosis. J. Neurosci. 19, 664-673.
Baccarini M. (2000) An old kinase on a new path: Raf and apoptosis. Cell Death Differ. 9, 783-785.
Barr R K, Bogoyevitch M A. (2001) The c-Jun N-terminal protein kinase family of mitogen-activated protein kinases (JNK MAPKs). Int J Biochem Cell Biol. 33, 1047-1063.
Bonni A, Brunet A, West A E, Datta S R, Takasu M A, Greenberg M E. (1999) Cell survival promoted by the Ras-MAPK signaling pathway by transcription-dependent and -independent mechanisms. Science 286, 1358-1362.
Bruckner S R, Tammariello S P, Kuan C Y, Flavell R A, Rakic P, Estus S. (2001) JNK3 contributes to c-Jun activation and apoptosis but not oxidative stress in nerve growth factor-deprived sympathetic neurons. J. Neurochem. 78, 298-303.
Bruckner S R, Estus S. (2002) JNK3 contributes to c-jun induction and apoptosis in 4-hydroxynonenal-treated sympathetic neurons. J Neurosci Res. 70, 665-670.
Brunet A, Datta S R, Greenberg M E. (2001) Transcription-dependent and -independent control of neuronal survival by the P13K-Akt signaling pathway. Curr Opin Neurobiol. 11, 297-305.
Chang L K, Putcha G V, Deshmukh M, Johnson E M Jr. (2002) Mitochondrial involvement in the point of no return in neuronal apoptosis. Biochimie. 84, 223-31.
Coffey E T, Smiciene G, Hongisto V, Cao J, Brecht S, Herdegen T, Courtney M J. (2002) c-Jun N-terminal protein kinase (JNK) 2/3 is specifically activated by stress, mediating c-Jun activation, in the presence of constitutive JNK1 activity in cerebellar neurons. J. Neurosci. 22, 4335-4345.
Copani A, Uberti D, Sortino M A, Bruno V, Nicoletti F, Memo M. (2001) Activation of cell-cycle-associated proteins in neuronal death: a mandatory or dispensable path? Trends Neurosci. 24, 25-31.
Crowder R J, Freeman R S. (1998) Phosphatidylinositol 3-kinase and Akt protein kinase are necessary and sufficient for the survival of nerve growth factor-dependentsympathetic neurons. J. Neurosci. 18, 2933-2943.
Datta S R, Dudek H, Tao X, Masters S, Fu H, Gotoh Y, Greenberg M E. (1997) Akt phosphorylation of BAD couples survival signals to the cell-intrinsic death machinery. Cell 91, 231-241.
Deshmukh M, Johnson E M Jr. (1997) Programmed cell death in neurons: focus on the pathway of nerve growth factor deprivation-induced death of sympathetic neurons. Mol Pharmacol. 51, 897-906
Dhillon A S, Kolch W. (2002) Untying the regulation of the Raf-1 kinase. Arch Biochem Biophys. 40, 3-9.
D'Mello S R, Galli C, Ciotti T, Calissano P. (1993) Induction of apoptosis in cerebellar granule neurons by low potassium: inhibition of death by insulin-like growth factor I and cAMP. Proc Natl Acad Sci USA. 90,10989-10993.
D'Mello S R, Borodezt K, Soltoff S P. (1997) Insulin-like growth factor and potassium depolarization maintain neuronal survival by distinct pathways: possible involvement of PI 3-kinase in IGF-1 signaling. J. Neurosci. 17, 1548-1560.
D'Mello S R. (1998) Molecular regulation of neuronal apoptosis. Curr Top Dev 187-213.
Dudek H, Datta S R, Franke T F, Birnbaum M J, Yao R, Cooper G M, Segal R A, Kaplan D R, Greenberg M E. (1997) Regulation of neuronal survival by the serine-protein threonine kinase Akt. Science 275, 661-665.
Duncia J V, Santella J B 3rd, Higley C A, Pitts W J, Wityak J, Frietze W E, Rankin F W, Sun J H, Earl R A, Tabaka A C, Teleha C A, Blom K F, Favata M F, Manos E J, Daulerio A J, Stradley D A, Horiuchi K, Copeland R A, Scherle P A, Trzaskos J M, Magolda R L, Trainor G L, Wexler R R, Hobbs F W, Olson R E. (1998) MEK inhibitors: the chemistry and biological activity of U0126, its analogs, and cyclization products. Bioorg Med Chem Lett. 8, 2839-2844.
Eilers A, Whitfield J, Babij C, Rubin LL, Ham J. (1998) Role of the Jun kinase pathway in the regulation of c-Jun expression and apoptosis in sympathetic neurons. J. Neurosci. 18, 1713-1724.
Estus S, Zaks W J, Freeman R S, Gruda M, Bravo R, Johnson E M Jr. (1994) Altered gene expression in neurons during programmed cell death: identification of c-jun as necessary for neuronal apoptosis. J. Cell Biol. 127, 1717-1727.
Favata M F, Horiuchi K Y, Manos E J, Daulerio A J, Stradley D A, Feeser WS, Van Dyk D E, Pitts W J, Earl R A, Hobbs F, Copeland R A, Magolda R L, Scherle P A, Trzaskos J M. (1998) Identification of a novel inhibitor of mitogen-activated protein kinase kinase. J Biol. Chem. 273, 18623-18632.
Foo S Y, Nolan G P (1999) NF-κB to the rescue: RELs, apoptosis and cellular transformation. Trends Genet. 15, 229-235.
Gonzalez-Polo R A, Mora A, Clemente N, Sabio G, Centeno F, Soler G, Fuentes J M. (2001) Mechanisms of MPP(+) incorporation into cerebellar granule cells. Brain Res Bull. 56,119-123.
Hall-Jackson C A, Eyers P A, Cohen P, Goedert M, Boyle F T, Hewitt N, Plant H, Hedge P. (1999) Paradoxical activation of Raf by a novel Raf inhibitor. Chem Biol. 6, 559-568.
Ham J, Babij C, Whitfield J, Pfarr C M, Lallemand D, Yaniv M, Rubin LL. (1995) A c-Jun dominant negative mutant protects sympathetic neurons against programmed cell death. Neuron 14, 927-939.
Han B H, Holtzman D M. (2000) BDNF protects the neonatal brain from hypoxic injury in vivo via the ERK pathway. J. Neurosci. 20, 5775-5781.
Harris C A, Deshmukh M, Tsui-Pierchala B, Maroney A C, Johnson E M Jr. (2002) Inhibition of the c-Jun N-terminal kinase signaling pathway by the mixed lineage kinase metabolism and growth of trophic factor-deprived neurons. J. Neurosci. 22, 103-113.
Hindley A, Kolch W. (2002) Extracellular signal regulated kinase (ERK)/mitogen activated protein kinase (MAPK)-independent functions of Raf kinases. J Cell Sci. 115, 1575-1581
Jansen B, Schlagbauer-Wadl H, Kahr H, Heere-Ress E, Mayer B X, Eichler H, Pehamberger H, Gana-Weisz M, Ben-David E, Kloog Y, Wolff K. (1999) Novel Ras antagonist blocks human melanoma growth. Proc Natl Acad Sci USA. 96, 14019-14024.
Jun T, Gjoerup O, Roberts T M. (1999) Tangled webs: evidence of cross-talk between C-Raf-1 and Akt. Sci STKE. 13, PE1.
Koulich E, Nguyen T, Johnson K, Giardina C, D'Mello S. (2001) NF-κB is involved in the survival of cerebellar granule neurons: association of IkappaBbeta phosphorylation with cell survival. J. Neurochem. 76, 1188-1198.
Kumari S, Liu X, Nguyen T, Zhang X, D'Mello S R. (2001) Distinct phosphorylation patterns underlie Akt activation by different survival factors in neurons. Brain Res Mol Brain Res. 96,157-162.
Kunimoto M. (1994) Methylmercury induces apoptosis of rat cerebellar neurons in primary culture. Biochem Biophys Res Commun. 204, 310-317.
Johnson E M and Deckworth T L. (1993) Molecular mechanisms of developmental neuronal death. Annu. Rev. Neurosci. 16, 31-46.
Lackey K, Cory M, Davis R, Frye S V, Harris P A, Hunter R N, Jung D K, McDonald O B, McNutt R W, Peel M R, Rutkowske R D, Veal J M, Wood E R. (2000) The discovery of potent cRaf1 kinase inhibitors. Bioorg Med Chem Lett. 10, 223-226.
Lin YZ, Yao SY, Veach RA, Torgerson TR, Hawiger J. (1995) Inhibition of nuclear translocation of transcription factor NF-κB by a synthetic peptide containing a cell membrane-permeable motif and nuclear localization sequence. J Biol. Chem. 270, 14255-14258
Liu DX, Greene L A. (2001) Neuronal apoptosis at the G1/S cell cycle checkpoint. Cell Tissue Res. 305, 217-228.
Linseman D A, Phelps R A, Bouchard R J, Le S S, Laessig T A, McClure M L, Heidenreich K A. (2002) Insulin-like growth factor-I blocks Bcl-2 interacting mediator of cell death (Bim) induction and intrinsic death signaling in cerebellar granule neurons. J. Neurosci. 22, 9287-9297.
Mattson M P. (2000) Apoptosis in neurodegenerative disorders. Nat Rev Mol Cell Biol. 1,120-129.
Mazzoni I E, Said F A, Aloyz R, Miller F D, Kaplan D. (1999) Ras regulates sympathetic neuron survival by suppressing the p53-mediated cell death pathway. J. Neurosci. 19, 9716-9727.
Miller T M, Tansey M G, Johnson E M Jr, Creedon D J. (1997a) Inhibition of phosphatidylinositol 3-kinase activity blocks depolarization- and insulin-like growth factor 1-mediated survival of cerebellar granule cells. J Biol. Chem. 272, 9847-9853.
O'Hare M, Wang F, Park D S. (2002) Cyclin-dependent kinases as potential targets to improve stroke outcome. Pharmacol Ther. 93,135-43.
Oppenheim R W (1991) Cell death during development of the nervous system. Annu. Rev. Neurosci. 14, 453-501.
Padmanabhan J, Park D S, Greene L A, Shelanski M L. (1999) Role of cell cycle regulatory proteins in cerebellar granule neuron apoptosis. J. Neurosci. 19, 8747-8756.
Park D S, Levine B, Ferrari G, Greene L A. (1997a) Cyclin dependent kinase inhibitors and dominant negative cyclin dependent kinase 4 and 6 promote survival of NGF-deprived sympathetic neurons. J. Neurosci. 17, 8975-8983.
Park D S, Morris E J, Greene L A, Geller H M. (1997b) G1/S cell cycle blockers and inhibitors of cyclin-dependent kinases suppress camptothecin-induced neuronal apoptosis. J. Neurosci. 17, 1256-1270.
Park D S, Morris E J, Padmanabhan J, Shelanski M L, Geller H M, Greene L A. (1998a) Cyclin-dependent kinases participate in death of neurons evoked by DNA-damaging agents. J. Cell Biol. 143, 457-467.
Park D S, Morris E J, Stefanis L, Troy C M, Shelanski M L, Geller H M, Greene L A. (1998b) Multiple pathways of neuronal death induced by DNA-damaging agents, NGF deprivation, and oxidative stress. J. Neurosci. 18, 830-840.
Pearson G, Bumeister R, Henry D O, Cobb M H, White M A. (2000) Uncoupling Raf1 from MEK1/2 impairs only a subset of cellular responses to Raf activation. J Biol. Chem. 275, 37303-37306.
Putcha G V, Moulder K L, Golden J P, Bouillet P, Adams J A, Strasser A, Johnson E M. (2001) Induction of BIM, a proapoptotic BH3-only BCL-2 family member, is critical for neuronal apoptosis. Neuron 29, 615-628.
Saporito M S, Hudkins R L, Maroney A C. (2002) Discovery of CEP-1347/KT-7515, an inhibitor of the JNK/SAPK pathway for the treatment of neurodegenerative diseases. Prog Med. Chem. 40, 23-62.
Trotter L, Panton W, Hajimohamadreza I, Petalidis L, Ward R, Fleming Y, Armstrong C G, Cohen P, Karran E H, Kinloch R A. (2002) Mitogen-activated protein kinase kinase 7 is activated during low potassium-induced apoptosis in rat cerebellar granule neurons. Neurosci Lett. 320, 29-32.
Vila M, and Przedborski S. (2003) Targeting programmed cell death in neurodegenerative diseases. Nat Rev Neurosci. 4, 365-375
Villalba M, Bockaert J, Journot L. (1997) Pituitary adenylate cyclase-activating polypeptide (PACAP-38) protects cerebellar granule neurons from apoptosis by activating the mitogen-activated protein kinase (MAP kinase) pathway. J. Neurosci. 17, 83-90.
Watson A, Eilers A, Lallemand D, Kyriakis J, Rubin L L, Ham J. (1998) Phosphorylation of c-Jun is necessary for apoptosis induced by survival signal withdrawal in cerebellar granule neurons. J. Neurosci. 18, 751-762.
Wiese S, Pei G, Karch C, Troppmair J, Holtmann B, Rapp U R, Sendtner M. (2001) Specific function of B-Raf in mediating survival of embryonic motoneurons and sensory neurons. Nat Neurosci. 4, 137-142
Weisz B, Giehl K, Gana-Weisz M, Egozi Y, Ben-Baruch G, Marciano D, Gierschik P, Kloog Y. (1999) A new functional Ras antagonist inhibits human pancreatic tumor Oncogene. 18, 2579-2588.
Weston C R, Davis R J. (2002) The JNK signal transduction pathway. Curr Opin Genet Dev. 12, 14-21.
Wolfman J C, Palmby T, Der C J, Wolfman A. (2002) Cellular N-Ras promotes cell survival by downregulation of Jun N-terminal protein kinase and p38. Mol Cell Biol. 22, 1589-1606.
Xu Z, Maroney A C, Dobrzanski P, Kukekov N V, Greene L A. (2001) The MLK family mediates c-Jun N-terminal kinase activation in neuronal apoptosis. Mol Cell Biol. 21, 4713-4724.

1. The use of a C-Raf inhibitor, or a pharmaceutically acceptable salt, complex or prodrug thereof, for the manufacture of a composition for the prevention or inhibition of neuronal cell death in a mammal suffering from or susceptible to neurodegenerative disease, cerebral ischaemia, traumatic neuronal injury, epilepsy-associated neuronal loss, paralysis, or spinal cord injury.

2. The use of a C-Raf inhibitor, or a pharmaceutically acceptable salt, complex or prodrug thereof, for the manufacture of a composition for the repair or regeneration of neuronal cells in a mammal.

3. The use of a C-Raf inhibitor, or a pharmaceutically acceptable salt, complex or prodrug thereof, for the manufacture of a composition for the prevention or inhibition of apoptotic neuronal cell death.

4. The use of a C-Raf inhibitor, or a pharmaceutically acceptable salt, complex or prodrug thereof, for the manufacture of a composition for the prevention or inhibition of neuronal cell death potentiated by inhibition or suppression of B-Raf.

5. The use of a C-Raf inhibitor, or a pharmaceutically acceptable salt, complex or prodrug thereof, for the manufacture of a composition for preventing or inhibiting neuronal cell death by stimulating or activating B-Raf.

6. The use of a C-Raf inhibitor as claimed in claim 3 wherein the composition is for the prevention or inhibition of neuronal cell death in a mammal suffering from or susceptible to neurodegenerative disease, cerebral ischaemia, traumatic neuronal injury, epilepsy-associated neuronal loss, paralysis or spinal cord injury.

7. The use of a C-Raf inhibitor as claimed in claim 4 wherein the composition is for the prevention or inhibition of neuronal cell death in a mammal suffering from or susceptible to neurodegenerative disease, cerebral ischaemia, traumatic neuronal injury, epilepsy-associated neuronal loss, paralysis or spinal cord injury.

8. The use of a C-Raf inhibitor as claimed in claim 5 wherein the composition is for the prevention or inhibition of neuronal cell death in a mammal suffering from or susceptible to neurodegenerative disease, cerebral ischaemia, traumatic neuronal injury, epilepsy-associated neuronal loss, paralysis or spinal cord injury.

9. The use as claimed in any one of claims 1 to 5, wherein the C-Raf inhibitor comprises an oxindole derivative, or a pharmaceutically acceptable salt, complex or prodrug thereof.

10. The use of claim 9, wherein said oxindole derivative further comprises {5-Iodo-3-[(3,5-dibromo-4-hydroxyphenyl) methylene]-2-indolinone}.

11. The use of claim 1, wherein said C-Raf inhibitor further comprises N-[5-(3-Dimethylaminobenzamido)-2-methylphenyl]-4-hydroxybenzamide.

12. A method of preventing or inhibiting neuronal cell death in a mammal suffering from or susceptible to neurodegenerative disease, cerebral ischaemia, traumatic neuronal injury, epilepsy-associated neuronal loss, paralysis, or spinal cord injury, comprising administering to the mammal an effective amount of a C-Raf inhibitor or a pharmaceutically acceptable salt, complex or prodrug thereof.

13. A method of repairing or regenerating neuronal cells in a mammal in need thereof, comprising administering to the mammal an effective amount of a C-Raf inhibitor or a pharmaceutically acceptable salt, complex or prodrug thereof.

14. A method of preventing or inhibiting apoptotic neuronal cell death in a mammal, comprising administering to the mammal an effective amount of a C-Raf inhibitor, or a pharmaceutically acceptable salt, complex or prodrug thereof.

15. The methods of claims 12 or 13 or 14, wherein said C-Raf inhibitor comprises {5-Iodo-3-[(3,5-dibromo-4-hydroxyphenyl) methylene]-2-indolinone}

16. A method of treating neurodegenerative disease, cerebral ischaemia, traumatic neuronal injury, epilepsy-associated neuronal loss, paralysis, or spinal cord injury, comprising administering to the mammal an effective amount of a B-Raf activator or a pharmaceutically acceptable salt, complex or prodrug thereof.


SEQUENCE LISTING (2 sequences)

SEQ ID NO: 1 - DNA, 13 bp, Artificial
  Oligonucleotide probe
gctggggactttc

SEQ ID NO: 2 - DNA, 22 bp, Artificial
  Oligonucleotide probe
attcgatcggggcggggcgagc